(19) United States
(12) Patent Application Publication
(10) Pub. No.: US 2021/0210159 A1
(43) Pub. Date: Jul. 8, 2021

(54) COMPUTATIONAL PROTEIN DESIGN USING TERTIARY OR QUATERNARY STRUCTURAL MOTIFS

(71) Applicant: Trustees of Dartmouth College, Hanover, NH (US)

(72) Inventors: Gevorg Grigoryan, Hanover, NH (US); Jianfu Zhou, West Lebanon, NH (US); Craig Mackenzie, Hopkinton, NH (US)

(21) Appl. No.: 17/059,060

(22) PCT Filed: May 30, 2019
(86) PCT No.: PCT/US19/34670

§ 371 Date: Nov. 25, 2020

Related U.S. Application Data
(60) Provisional application No. 62/678,588, filed on May 31, 2018.

(51) Int. Cl. G16B 15/30; G16B 30/00; C12P 21/02

(52) U.S. Cl. CPC G16B 15/30; C12P 21/02; G16B 30/00

(57) ABSTRACT

This disclosure relates to a method for constructing an amino acid sequence or a library of amino acid sequences capable of folding into pre-defined structure or into a binding partner of a target structure. The method is based on the concept that protein structure space is modular, composed of highly recurrent structural building blocks.

### CROSS REFERENCE TO RELATED APPLICATIONS

This patent application is a National Stage Entry of International Patent Application No. PCT/US2019/034670, filed on May 30, 2019, which claims priority to U.S. Provisional Patent Application No. 62/678,588, filed on May 31, 2018, the entire contents of which are fully incorporated herein by reference.

### FEDERALLY SPONSORED RESEARCH OR DEVELOPMENT

This invention was made with Government support under DMR1534246 awarded by the National Science Foundation and P20 GM113132 awarded by the National Institutes of Health. The Government has certain rights in this invention.

### TECHNICAL FIELD

The present disclosure relates to computational protein design and, in particular, to methods, devices, and systems for designing a protein that can fold into a pre-defined structure or the binding partner of a target structure.

### BACKGROUND

Computational protein design (CPD) is the task of finding amino-acid sequences that fold into a pre-defined structure (the target). The basic idea behind the modern approach to CPD, which was initially formulated in the mid-1990s, is to capture the amino-acid sequence determinants of basic protein phenomena (e.g., folding and binding) from physical principles. Specifically, the aim is to approximate the free energy of any protein sequence in the target structure by modeling the underlying inter-atomic interactions. A computational procedure for doing so is referred to as a scoring function. With a scoring function in hand, one can perform CPD by looking for sequences that have particularly favorable energies for a given target.

In practice, many issues limit the accuracy of traditional CPD, ultimately leading to low robustness. It is presently infeasible to model the physics of protein structure at a sufficient level of detail to compute accurate free energies in the context of design. Thus, significant approximations must be made in physics-based scoring functions that strongly limit their predictive ability. As an alternative, some basic physical phenomena can be modeled empirically through knowledge-based potentials (also known as statistical potentials). With these, instead of evaluating the energetics of atomic interactions to derive the favorability of specific structural features (e.g., two specific atoms being at a particular distance from each other), one measures the frequencies of these features in known protein structures and quantifies their empirical favorability by assuming that the more frequent ones are more favorable. For example, simple structural features such as backbone dihedral angles, atomic distances and packing densities, bond orientations, residue burial states, and inter-residue contacts, have been exploited to build statistical potentials. Whether one relies of a physics-based, statistical, or a hybrid energy function, the fundamental problem of CPD remains: although the details of inter-atomic interactions really do ultimately shape sequence-structure relationships (i.e., which sequences will fold into a given structure), they are nevertheless very many steps removed from these relationships. Thus, even small amount of error in modeling atomistic phenomena can compound to significant errors in the ultimate prediction of amino-acid sequences. This is made worse by the fact that errors in existing potentials are not small and not random; rather, they are large and systematic, associated with often entirely missing contributions, such as configurational entropy, free energy of the unfolded state, or the presence of solvent. Indeed, even the basic assumption that elementary inter-atomic interactions and other energetic contributions are additive is merely an approximation. For example, it is known that the free energy of a protein sequence in a given configurational ensemble is not an additive function of its inter-atomic interactions, particularly when considering the effect of the solvent.

Thus, there is a need in the art for an approach to protein design that provides a new way of addressing the scoring function problem in a way that leads to significantly higher success rates of CPD.

### SUMMARY OF THE INVENTION

The present disclosure provides a new CPD method based on observing sequence-to-structure relationships directly, from existing protein structures, rather than deriving them indirectly by modeling the underlying atomistic physics. Protein structure represents a quasi-discrete space in which only certain backbone geometries are allowed (i.e., are designable) in the sense that they can be realized with a sequence of natural amino acids. Local backbone structural motifs around each residue in the Protein Data Bank (PDB), which capture secondary, tertiary, and quaternary structural contexts, have been systematically characterized (1). These motifs, which are collectively referred to herein as “TERMs” (short for tertiary motifs, though, as mentioned above these motifs capture secondary, tertiary, and quaternary structures), are highly reused in nature, across unrelated proteins. For example, only ˜600 TERMs are sufficient to describe 50% of the known structural universe at sub-A resolution (1). By virtue of this apparent degeneracy of structure space, TERMs effectively capture fundamental rules of sequence-structure relationships. This is because each motif occurs many times in the PDB, often in thousands of different sequence/structure contexts. By analyzing the sequences of these many matches, one can extract the sequence determinants of the structural fragment represented by the corresponding TERM.

There are at least three advantages of the approach provided herein over the state of the art. First, the method described herein designs sequences based on the proven rules of sequence-structure relationships observed in native proteins. That is, one knows a priori that the sequence of every TERM match considered toward the design procedure really does form the corresponding backbone conformation, which is a part of the target structure. This type of design from known building blocks means that one can expect much higher success rates than those of existing methods (this has been observed in validation studies disclosed herein). Second, in relation to statistical scoring functions, which are also based on existing protein structures, the method described herein does not assume additivity and independence between the preferences of elementary structural features such as distances and angles. Instead, by directly observing TERM-based sequence-structure preferences, the method (implicitly) accounts for the collective action of multiple contributions. Finally, a TERM-based approach offers a novel way of recognizing that proteins are not static molecules, but exist as conformational ensembles at room temperature. This is because sequence statistics (and ultimately the scoring function) arise from structural ensembles represented by TERM matches—close, but not exact instances of similar backbone configurations found in a structural database (e.g., a structural database comprising native proteins). Thus, TERM-based design enables identification of an amino acid sequence that is compatible not only with the specified frozen backbone configuration, but also with an ensemble of close configurations, which is a more appropriate representation of a protein structural state. Approaches that address the need to model backbone flexibility have been proposed in the context of existing CPD methods, but they are subject to the same limitations of scoring accuracy (and ultimately robustness) discussed in the Background section, in addition to incurring significant computational cost.

In one aspect, this disclosure provides an approach to protein design based on obtaining sequence statistics in the context of holistic atomistically-defined structural environments. This approach is advantageous at least because it avoids having to assume additivity of elementary structural descriptors, but also recognizes and takes advantage of the natural degeneracy of protein structure. Indeed, the superior performance of this approach can, at least in part, be attributed to its recognition that the protein structural universe represents a quasi-discrete space, in which only certain backbone geometries are allowed (i.e., are designable). Thus, this disclosure provides an approach to protein design that leverages the statistics of precisely-defined detailed structural environments.

In another aspect, this disclosure provides methods for in silico design of an amino acid sequence. In certain embodiments, the methods comprise the steps of decomposing the target structure into a plurality of structural motifs; identifying, in a structural database, a plurality of structural matches for each of the plurality of structural motifs; deducing a value for at least one non-local energetic contribution to a sequence-structure relationship using each of the plurality of structural matches; and generating at least one candidate amino acid sequence. In certain embodiments, the candidate amino acid sequence possesses a designable property. In certain embodiments, the candidate amino acid sequence is a protein that is foldable into a binding partner of the target structure. In certain embodiments, the at least one non-local energetic contribution is from a contiguous stretch of backbone around a single design position (e.g., (i−n) through (i+n), where i is a given position and n is a controllable parameter) within one of the plurality of structural motifs. In certain embodiments, the at least one non-local energetic contribution is from a backbone in spatial but not sequence proximity to a single design position within one of the plurality of structural motifs. In certain embodiments, the at least one non-local energetic contribution is from a pair of coupled residues within one of the plurality of structural motifs. In certain embodiments, the methods further comprise the step of acquiring a value for at least one local energetic contribution to a sequence-structure relationship using each of the plurality of structural matches. In some such embodiments, the at least one local energetic contribution is from a backbone angle for a single design position within one of the plurality of structural motifs. In some such embodiments, the backbone angle is a phi, psi, or omega angle. In certain embodiments, the target structure is a tertiary structure of a protein. In certain embodiments, the target structure is a quaternary structure of a protein complex.

In yet another aspect, this disclosure provides methods for in silico design of an amino acid sequence. In certain embodiments, the methods comprise the steps of: decomposing the target structure into a plurality of structural motifs; identifying, in a structural database, a plurality of structural matches for each of the plurality of structural motifs; sequentially deducing a set of values for energetic contributions to a sequence-structure relationship using each of the plurality of structural matches according to a hierarchy of energetic contributions, the hierarchy comprising at least two of: (i) at least one local energetic contribution for a single design position within one of the plurality of structural motifs, (ii) a contiguous stretch of backbone around the single design position, (iii) a backbone in spatial but not sequence proximity to the single design position, and (iv) a pair of coupled residues comprising the single design position; and generating at least one candidate amino acid sequence. In certain embodiments, the candidate amino acid sequence is a protein that is foldable into a binding partner of the target structure. In certain embodiments, the hierarchy further comprises a higher order contribution. In certain embodiments, the hierarchy further comprises (v) a triplet of residues comprising the single design position. In certain embodiments, the at least one local energetic contribution is from a backbone angle for a single design position within one of the plurality of structural motifs. In certain embodiments, the at least one local energetic contribution is from a burial state of a single design position within one of the plurality of structural motifs. In certain embodiments, the target structure is a tertiary structure of a protein. In certain embodiments, the target structure is a quaternary structure of a protein complex.

In yet another aspect, this disclosure provides non-transitory computer-readable storage media encoded with instructions for in silico design of an amino acid sequence that can fold into a binding partner of the target structure. The instructions are executable by a processor and comprise the methods disclosed herein.

In still another aspect, this disclosure provides methods for making a protein that folds into a binding partner of a target structure. In certain embodiments, the method comprises providing a nucleic acid sequence encoding a candidate amino acid sequence generated by the in silico design methods disclosed herein; introducing the nucleic acid sequence into a host cell; and expressing the candidate amino acid sequence. In certain embodiments, the methods further comprise determining whether the candidate amino acid sequence folds into a binding partner of the target structure.

In still another aspect, this disclosure provides proteins produced by the methods disclosed herein.

In certain embodiments for any of the aspects described herein, the protein is selected from the group consisting of an enzyme, antibody, receptor, transport protein, hormone, growth factor, and a fragment thereof.

In certain embodiments for any of the aspects described herein, the protein is a designed variant of a target structure. In some such embodiments, the target structure is selected from the group consisting of a fluorescent protein, a G protein-coupled receptor (GPCR), and a protein containing a PDZ domain.

In certain embodiments for any of the aspects described herein, the target structure is a fluorescent protein. In some such embodiments, the fluorescent protein is red fluorescent protein (RFP).

In certain embodiments for any of the aspects described herein, the target structure is a G protein-coupled receptor (GPCR). In some such embodiments, the GPCR is an adrenergic receptor such as beta-1 adrenergic receptor.

In certain embodiments for any of the aspects described herein, the target structure is a protein containing a PDZ domain. In some such embodiments, the protein containing a PDZ domain is Na⁺/H⁺ exchanger regulatory factor 2 (NHERF-2) (also called E3KARP, SIP-1, and TKA-1). In some such embodiments, the protein containing a PDZ domain is membrane-associated guanylate kinase (MAGI-3).

In certain embodiments for any of the aspects described herein, the binding partner of the target structure is a protein or other molecule that binds to a PDZ domain. In some such embodiments, the binding partner of the target structure is lysophosphatidic acid receptor 2 (LPA2).

These and other objects of the invention are described in the following paragraphs. These objects should not be deemed to narrow the scope of the invention.

### BRIEF DESCRIPTION OF THE DRAWINGS

For a better understanding of the invention, reference may be made to embodiments shown in the following drawings.

FIG. 1 shows a flowchart according to an exemplary embodiment of the present technology.

FIGS. 2A and 2B show a flowchart according to an exemplary embodiment of the present technology.

FIG. 3 shows a flowchart according to an exemplary embodiment of the present technology.

FIG. 4 is a schematic representation of an exemplary computational protein design method.

FIG. 5 shows the total surface redesign of an exemplary target structure, mCherry. The left panel shows, as gray spheres, the 64 surface positions that were allowed to vary in design. The middle and right panels show the surface of the original mCherry and the redesigned variant, respectively, with the vacuum electrostatic potential designated with false color.

FIG. 6 shows size-exclusion chromatograms of mCherry proteins. The top panel shows the chromatogram of a standard, containing the wild-type mCherry and a mCherry-LOV2 fusion protein (the latter as described by Wang et al. (2)). The bottom panel shows the chromatogram of the redesigned mCherry variant by itself, showing it to elute at close to the same volume as the wild type. Based on the standards, the dimeric protein would be expected to elute at the volume indicated by a dotted line, which eliminates the possibility of design oligomerization. Thus, size-exclusion chromatography shows the designed mCherry protein to be monomeric in solution.

FIG. 7 shows absorbance spectra of mCherry proteins. The top panel compares absorbance spectra of wild-type and redesigned mCherry proteins (with absorbance values shown on the left and right Y-axes, respectively), showing the two exhibit similar spectral shapes. The bottom panel compares fluorescence spectra of the two proteins, measured at equivalent protein concentrations. The redesigned mCherry protein preserves photo properties of the fluorophore.

FIG. 8 shows the chemical denaturation of mCherry and an exemplary designed variant. Degree of foldedness was monitored via chromophore absorbance at 587 nm. Because the chromophore rapidly hydrolyzes upon exposure to water, this constitutes a sensitive metric of structure. Data are fit to the Hill equation, with the concentration of half denaturation noted in the legend.

FIG. 9 shows the crystal structure of β1 adrenergic receptor GPCR (PDB entry 4BVN), with red and blue lines indicating the approximate locations of extracellular and cytoplasmic membrane boundaries (left panel). The middle and right panels show in-vacuo electrostatic surface potentials of the wild-type GPCR and its redesigned counterpart, respectively (in the same orientation).

FIG. 10A-10D illustrate the four different topologies that Baker and co-workers targeted in their design study (3). FIG. 10E-10F show the correlation between the length-normalized score of each design (on its respective backbone) on the X-axis, computed using an exemplary design method described herein, and the experimentally-derived stability score for each sequence on the Y-axis. Point color in the scatter plot indicates data density, with red being the densest and blue the least dense. The mean curve is shown with a black line with circles, obtained by averaging the stability score in ten progressive windows of the score. FIG. 10I-10L show the same plots as in FIG. 10E-10F, respectively, but with a score computed using the Rosetta method on the X-axis. In each case, the correlation exhibited by a score computed using an exemplary design method disclosed herein significantly exceeds that for a score computed using Rosetta. In fact, in three out of the four cases for Rosetta, the correlation is either of the wrong sign or is statistically insignificant (panels indicated by “X”). While the correlation is always of the right sign and statistically highly significant for the exemplary design methods disclosed herein (as indicated by black checkmarks). Thus, statistical energy computed by the TERM-based methods disclosed herein indicates design quality.

FIG. 11A-11D correspond to variants of human Pin1 WW domain (modeled using PDB entry 2ZQT), human Yes-associated protein 65 WW domain (modeled using PDB entry 4REX), villin headpiece helical subdomain (residues 42-76; modeled using PDB entry 1VII), and peripheral subunit-binding domain family member BBL (modeled PDB entry 2WXC), respectively. Each data point corresponds to a single sequence variant, with its thermodynamic stability plotted against its score computed using an exemplary design method described herein. Thermodynamic stability is represented by the free energy of unfolding in FIGS. 11A, 11C, and 11D, and apparent melting temperature in FIG. 11B). Best-fit lines are produced using robust linear regression with bisquare weighting function. The Pearson correlation is show in the title for each panel. Outlier points, identified using the Tukey fences approach, are labeled with a red outline and not included in calculating correlation coefficients. Thus, scores computed by the TERM-based methods disclosed herein correlate with thermodynamic stability.

FIG. 12 shows the procedure for designing a novel PDZ binding mode. In all panels, N2P2 is shown in green and the binding peptide (from PDB entry 2HE4) in black. FIG. 12A shows a completing TERM (cyan sticks), with one segment overlapping with the binding peptide and another forming contacts with N2P2 surface regions outside of the binding pocket (contacting positions labeled in red). FIG. 12B shows multiple means of connecting the completing TERM with the original binding peptide using other TERMs in the library. FIG. 12C shows the final backbone template and with the designed sequence.

FIG. 13 shows plots from an FP-based inhibition assay of designed peptide against N2P2 (left) and M3P6 (right). Inhibition constants are shown on the plots.

FIG. 14A shows a backbone of the de novo-designed structures targeted by Rocklin et al. (3). FIG. 14B shows a structural model of the sequence designed using the exemplary design methods disclosed herein for this backbone (sequence shown on the bottom). All 40 positions were allowed to take on any natural amino acid. FIG. 14C shows superposition between the target backbone (green) and the experimentally-determined structure of the corresponding design by Baker and co-workers (cyan) (3). This structure (PDB code 5UP5) is the top hit for the designed sequence produced by the structure-prediction method HHPred (4). The second hit is the PDB entry 1UTA, whose relevant portion (cyan) is shown superimpose onto the target backbone (green) in FIG. 14D). Thus, the exemplary design methods disclosed herein can be applied to design structures generated de novo.

### DETAILED DESCRIPTION OF THE INVENTION

This detailed description is intended only to acquaint others skilled in the art with the present invention, its principles, and its practical application so that others skilled in the art may adapt and apply the invention in its numerous forms, as they may be best suited to the requirements of a particular use. This description and its specific examples are intended for purposes of illustration only. This invention, therefore, is not limited to the embodiments described in this patent application, and may be variously modified.

In at least one aspect, this disclosure provides methods for designing an amino acid sequence. The methods comprise deducing a value for at least one non-local pseudo-energetic contribution from structural matches to an appropriately defined structural motif (i.e., a backbone fragment excised from the structure, comprising one or more disjoint backbone segments), such as a tertiary structural motif or a quaternary structural motif, of the target structure. In certain embodiments, the designed amino acid sequence is a protein that folds into a binding partner of the target structure.

In certain embodiments, the non-local pseudo-energetic contribution is an own-backbone contribution, a near-backbone contribution, a pair contribution, and/or a triplet (or higher-order) contribution.

In certain embodiments, the value for the non-local pseudo-energetic contribution is deduced from sequence statistics of the structural matches. In a preferred embodiment, sequence statistics within a structural match are driven by amino acid positions contained within the structural motif (e.g., a pair of amino acids influences the sequence statistics if and only if the corresponding pair of positions are contained within the structural motif).

In certain embodiments, the structural match is obtained by querying a structural database. In some such embodiments, the structural database is the Protein Data Bank (PDB). In other such embodiments, the structural database is a specialized database containing, for example, only transmembrane proteins.

In certain embodiments, the target structure is decomposed into a plurality of structural motifs. In some such embodiments, the target structure is a protein and the structural motifs comprise secondary and tertiary structural motifs. In some such embodiments, the target structure is a protein complex and the structural motifs comprise secondary, tertiary, and/or quaternary structural motifs. In certain embodiments, the structural motif for a given residue, i, of a target structure comprises the own-backbone (e.g., residues i−2 to i+2) and the near backbone (e.g., backbone around all residues with which i is capable of forming contacts).

In certain embodiments, the method further comprises deducing values for at least one local pseudo-energetic contribution from structural matches. In some such embodiments, the local pseudo-energetic contribution is a contribution from a dihedral angle and/or the burial state of a given amino acid residue, i. Thus, in certain embodiments, the method comprises deducing a set of values for each of a non-local pseudo-energetic contribution and a local pseudo-energetic contribution. In some such embodiments, the pseudo-energetic contributions are deduced according to a hierarchy: (1) local pseudo-energetic contribution(s) and (2) non-local pseudo-energetic contribution(s). For example, the hierarchy may comprise at least two of: (i) at least one local pseudo-energetic contribution for a single amino-acid residue (e.g., a given residue, i) within the structural match, (ii) a contiguous stretch of backbone around the single amino-acid residue (e.g., (i−n) through (i+n), where i is a given position and n is a controllable parameter), (iii) a backbone in spatial but not sequence proximity to the single amino-acid residue (e.g., backbone around all residues with which i is capable of forming contacts), and/or (iv) a pair of coupled residues comprising the single design position. As another example, the hierarchy may comprise pseudo-energetic contributions from: (i) a backbone dihedral angle, such as the phi angle, psi angle, and/or omega angle, for an amino acid in a particular design position of the target structure, (ii) a burial state of the amino acid in the particular design position, (iii) a contiguous stretch of backbone around the single amino acid residue, (iv) a backbone in spatial but not sequence proximity to the design position, and/or (v) a pair of coupled residues comprising the amino acid in the design position. By including higher-order contributions later in the hierarchy, such contributions are only used as correctors (and only to the extent necessary) over what is already described by lower-order contributions. In this way, pseudo-energetic contributions are considered in a hierarchy, with each next type of contribution introduced only to describe what is not already captured by previous ones. In certain embodiments, hierarchical consideration of local and non-local contributions is beneficial because the earliest contributions in the hierarchy are those associated with the strongest sequence statistics, such that highest-confidence effects are captured first, relatively unaffected by statistical noise.

In a preferred embodiment, higher-order pseudo-energetic contributions are considered only as needed (i.e., models involving only lower-order pseudo-energetic contributions are preferred to those also involving higher-order contributions, if they equally describe the observations). In some such embodiments, higher-order pseudo-energetic contributions act as correctors to lower-order contributions. For example, pair energies are needed only to describe those aspects of sequence statistics that are not satisfactorily described with self contributions.

In the various aspects disclosed herein, protein design based on structural motifs, particularly tertiary and/or quaternary structural motifs, enables the selection of an amino acid sequence that is compatible not only with the frozen backbone configuration of the target structure, but also with an ensemble of close configurations—the appropriate representation of a protein structural state.

### A. COMPUTATIONAL PROTEIN DESIGN

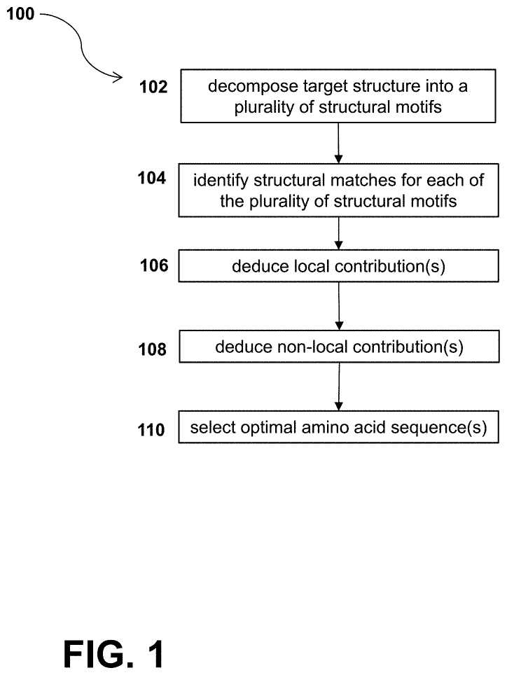

FIG. 1 shows a flow diagram of a method 100 for designing an amino acid sequence, such as, for example, a protein that folds into a binding partner of a target structure. As shown at box 102, a target structure is decomposed into a plurality of secondary, tertiary, or quaternary structural motifs. Such decomposition may be guided by a graph representation of (i) the target structure's coupled residues and/or (ii) the target structure's residue-backbone influences. For example, each secondary, tertiary, or quaternary structural motif is formed around a set of one or more amino acid residues that represent a connected sub-graph of the graph representing the target structure's coupled residues. In certain embodiments, the target structure is decomposed into as few tertiary (or quaternary) structural motifs needed to describe the target structure.

As shown at box 104, once a tertiary (or quaternary) structural motif has been identified, a structural database is queried to identify structural matches. The structural database may be, for example, the entire PDB or a filtered subset of the PDB. The structural database may be stored in a local and/or a remote memory, for example. The data stored in the structural database may be in any suitable format. In certain embodiments, a search engine, such as MASTER, is employed to query the structural database. In certain embodiments, the search engine takes as a query a secondary, tertiary (or quaternary) structural motif and returns all of fragments from a structural database matching the query to within a given root mean squared deviation (RMSD) threshold. The result set, which contains structural matches, may be ordered, such as by increasing RMSD.

At box 106, local pseudo-energetic contribution(s) are deduced. A local pseudo-energetic contribution may be associated with a backbone dihedral angle (i.e., the phi angle, psi angle, or omega angle) for a single amino acid at a given position in the target or the burial state of a single amino acid at a given target position. The local pseudo-energetic contribution may be deduced from sequence statistics of corresponding structural environments within the PDB.

At box 108, non-local pseudo-energetic contribution(s) are deduced. A non-local pseudo-energetic contribution may be associated with a contiguous stretch of backbone around a single design position, a backbone in spatial but not sequence proximity to the single design position, and/or a pair of coupled residues comprising the single design position. The non-local pseudo-energetic contribution may be deduced from sequence statistics of structural matches to appropriately constructed TERMs.

At box 110, an optimal amino acid sequence or set of amino acid sequences is selected. A variety of optimization methods can be used to select the optimal amino acid sequence or set of amino acid sequences. For example, an Integer Linear Programming (ILP) approach, which allows for the introduction of constraints into the design problem (e.g., sequence symmetry constraints, or constraints on the number of charged/polar residues, or limits on the residues mutated relative to some starting sequence, etc.), may be used. As another example, Self-Consistent Mean Field (SCMF) or Belief Propagation (BP) techniques may be used. As still another example, Simulated Annealing Monte Carlo (MC) may be used.

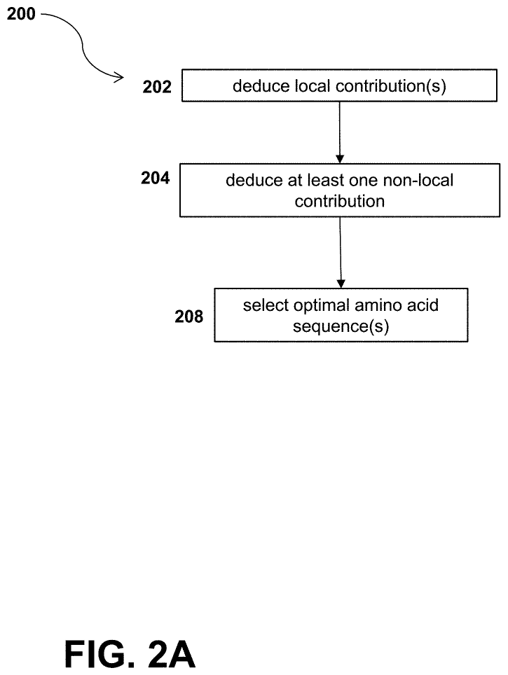

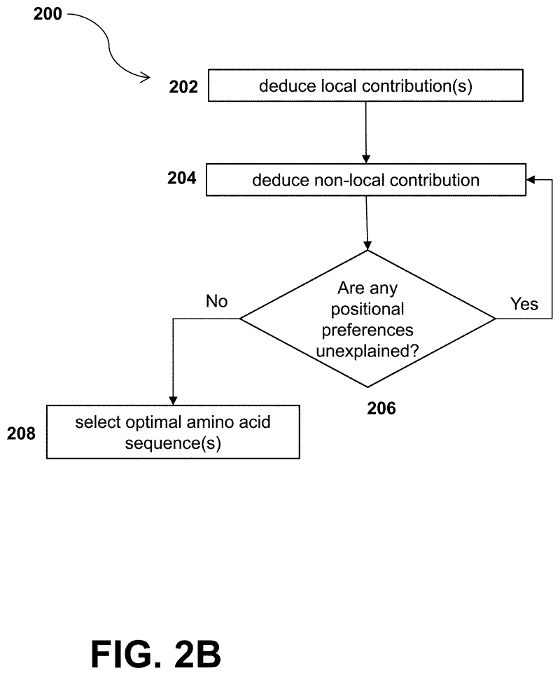

FIG. 2A shows a flow diagram of a method 200 for deducing pseudo-energetic contributions from sequence statistics of the structural matches and environments.

At box 202, local pseudo-energetic contribution(s) are deduced. A local pseudo-energetic contribution may be from a backbone angle, such as the phi angle, psi angle, and/or omega angle, for a single design position within the structural match and/or a burial state of the single design position. The local pseudo-energetic contribution may be deduced from sequence statistics of the structural matches.

At box 204, at least one non-local pseudo-energetic contribution is deduced. For example, the at least one non-local pseudo-energetic contribution may be from a contiguous stretch of backbone around a single design position.

Subsequent non-local pseudo-energetic contributions may be deduced as indicated by block 204. The subsequent non-local pseudo-energetic contribution may be, for example, a backbone in spatial but not sequence proximity to the single design position, a pair of coupled residues comprising the single design position, and/or a triplet of residues comprising the single design position.

An optimal amino acid sequence or set of amino acid sequences is selected as indicated by block 208. A variety of optimization methods can be used to select the optimal amino acid sequence or set of amino acid sequences, including, but not limited to an ILP, SCMF, BP, or MC approach, as described above.

In certain embodiments, such as depicted in FIG. 2A, a plurality of non-local pseudo-energetic contributions are deduced, as indicated by block 204. For example, the plurality of non-local pseudo-energetic contributions may be from (i) a contiguous stretch of backbone around a single design position, (ii) a backbone in spatial but not sequence proximity to the single design position, (iii) a pair of coupled residues comprising the single design position, and/or (iv) a triplet of residues comprising the single design position. In some such embodiments, each of the aforementioned contributions (i)-(iv) are calculated in the order specified. However, in such embodiments, the subsequent contributions only have to explain the difference between what is already explained and observed. Thus, subsequent contributions in the hierarchy will likely get progressively smaller and may even approach insignificance if there is not much left to describe. For example, subsequent contributions may end up being zero or substantially zero, in which case it almost as if they were not calculated.

FIG. 2B shows a flow diagram of a method 200 for deducing pseudo-energetic contributions from sequence statistics of the structural matches and environments.

At box 202, local pseudo-energetic contribution(s) are deduced. A local pseudo-energetic contribution may be from a backbone angle, such as the phi angle, psi angle, and/or omega angle, for a single design position within the structural match and/or a burial state of the single design position. The local pseudo-energetic contribution may be deduced from sequence statistics of the structural matches.

At box 204, a first non-local pseudo-energetic contribution is deduced. For example, the first non-local pseudo-energetic contribution may be from a contiguous stretch of backbone around a single design position.

As indicated by decision diamond 206, alternative responses occur depending upon whether any positional preferences remain unexplained. If a positional preference is unexplained, a subsequent non-local pseudo-energetic contribution is deduced as indicated by block 204. The subsequent non-local pseudo-energetic contribution may be, for example, a backbone in spatial but not sequence proximity to the single design position, a pair of coupled residues comprising the single design position, and/or a triplet of residues comprising the single design position. If a positional preference does not remain unexplained, an optimal amino acid sequence or set of amino acid sequences is selected as indicated by block 208. A variety of optimization methods can be used to select the optimal amino acid sequence or set of amino acid sequences, including, but not limited to an ILP, SCMF, BP, or MC approach, as described above.

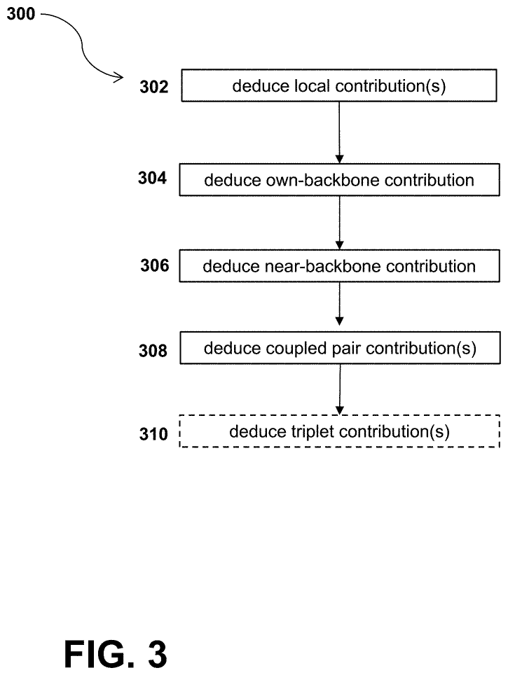

FIG. 3 shows a flow diagram of a method 300 for deducing pseudo-energetic contributions from sequence statistics of the structural matches and matching environments.

At box 302, local pseudo-energetic contribution(s) are deduced. A local pseudo-energetic contribution may be from a backbone angle, such as the phi angle, psi angle, and/or omega angle, for a single design position within the structural match and/or a burial state of the single design position. The local pseudo-energetic contribution may be deduced from sequence statistics of the structural matches. At box 304, a non-local pseudo-energetic contribution from a contiguous stretch of backbone around a single design position (i.e., an own-backbone contribution) is deduced. At box 306, a non-local pseudo-energetic contribution from a backbone in spatial but not sequence proximity to the single design position (i.e., a near-backbone contribution) is deduced. At box 308, a non-local pseudo-energetic contribution from a pair of coupled residues comprising the single design position (i.e., a coupled pair contribution) is deduced. At box 310, a non-local pseudo-energetic contribution from a triplet of residues comprising the single design position (i.e., a triplet or other higher order contribution) is optionally deduced.

In this way, pseudo-energetic contributions are deduced in a hierarchy, with each next type of contribution introduced only to describe what is not already captured by previous ones.

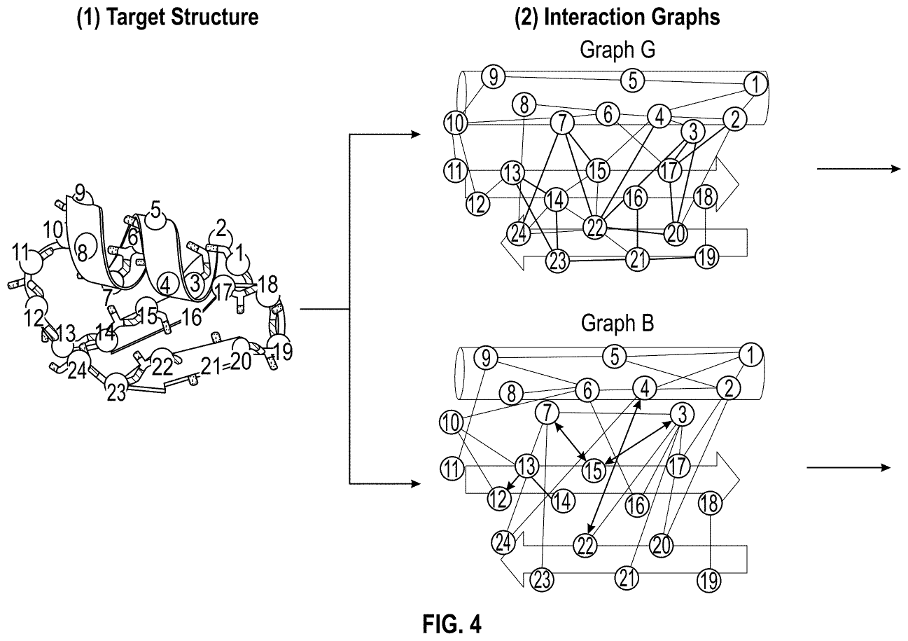

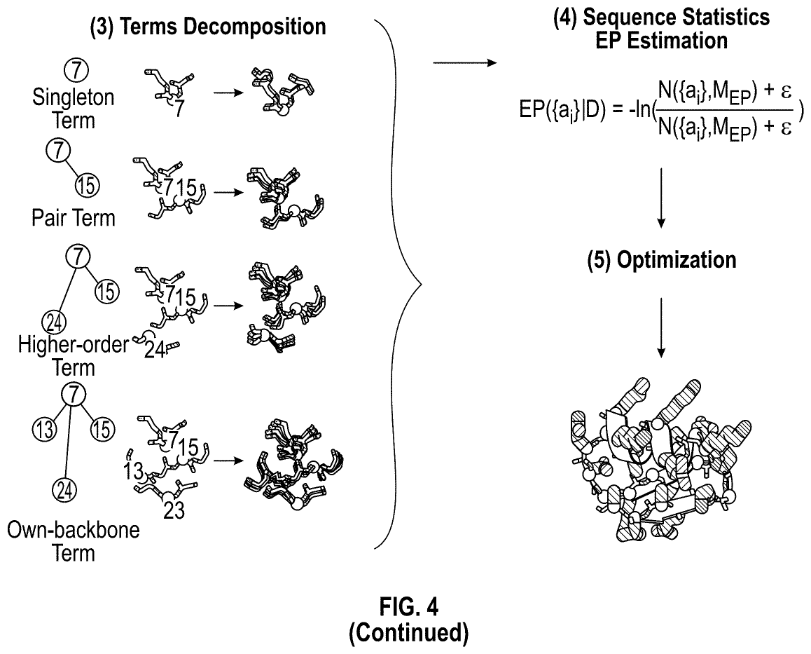

FIG. 4 shows a schematic representation of an exemplary computational protein design method based on tertiary/quaternary structural motifs. As depicted in FIG. 4, a target structure may be decomposed into secondary/tertiary/quaternary structural motifs guided by a graph representation of (a) its coupled residues, shown as Graph G, and (b) the residue-backbone influences, shown as Graph B. Structural matches to each structural motif may be identified from a structural database. Sequence alignments implied by the structural matches may be used to derive values for pseudo-energetic contributions that govern the sequence-structure relationship in the target structure. Given values for pseudo-energetic contributions, combinatorial optimization may be used to produce an optimal amino acid sequence or a library of optimal amino acid sequences.

In certain embodiments, at least a portion of the activity described with respect to FIGS. 1-4 may be implemented via one or more application-specific integrated circuits (ASICs), field programmable gate arrays (FPGAs), discrete logic, and/or using software executable by one or more servers or computers, such as a computing device with a processor and a memory. The processor can be any custom made or commercially available processor, such as, for example, a Core series, vPro, Xeon, or Itanium processor made by Intel Corporation, or a Phenom, Athlon, Sempron, or Opteron-series processor made by Advanced Micro Devices, Inc. The processor may also represent multiple parallel or distributed processors working in unison.

The software in the memory may include one or more separate programs or applications. The programs may have ordered listings of executable instructions for implementing logical functions. The software may include a suitable operating system of the servers or computers, such as macOS, OS X, Mac OS X, and iOS from Apple, Inc.; Windows, Windows Phone, and Windows 10 Mobile from Microsoft Corporation; a Unix operating system; a Unix-derivative (e.g., BSD or Linux); and Android from Google, Inc. The operating system essentially controls the execution of other computer programs, and provides scheduling, input-output control, file and data management, memory management, and communication control and related services.

In general, a computer program product or computer-readable storage medium in accordance with the embodiments includes a computer usable storage medium (e.g., standard random access memory (RAM), an optical disc, a universal serial bus (USB) drive, or the like) having computer-readable program code embodied therein, wherein the computer-readable program code is adapted to be executed by the processor (e.g., working in connection with an operating system) to implement the methods described below. In this regard, the program code may be implemented in any desired language, and may be implemented as machine code, assembly code, byte code, interpretable source code or the like (e.g., via C, C++, Java, Actionscript, Objective-C, Javascript, CSS, XML, and/or others).

The memory can include any one or a combination of volatile memory elements (e.g., random access memory (RAM, such as DRAM, SRAM, SDRAM, etc.)) and nonvolatile memory elements (e.g., ROM, hard drive, flash drive, CDROM, etc.). It may incorporate electronic, magnetic, optical, and/or other types of storage media. The memory can have a distributed architecture where various components are situated remote from one another, but are still accessed by the processor. These other components may reside on devices located elsewhere on a network or in a cloud arrangement.

The servers or computers may include a transceiver that sends and receives data over a network, for example. The transceiver may be adapted to receive and transmit data over a wireless and/or wired (e.g., Ethernet) connection. The transceiver may function in accordance with the IEEE 802.11 standard or other standards. More particularly, the transceiver may be a WWAN transceiver configured to communicate with a wide area network including one or more cell sites or base stations to communicatively connect the servers or computers to additional devices or components. Further, the transceiver may be a WLAN and/or WPAN transceiver configured to connect the servers or computers to local area networks and/or personal area networks, such as a Bluetooth network.

A1. Target Structure Decomposition and Identifying Structural Matches

In at least one aspect, this disclosure provides a method for computational protein design, the method comprising decomposing a target structure into a plurality of structural motifs. In certain embodiments, the target structure is a tertiary structure of a protein. In certain embodiments, the target structure is a quaternary structure of a protein complex.

In certain embodiments, the plurality of structural motifs covers each residue and each pair of coupled residues in the target structure. For example, every residue and every pair of couple residues may be covered by at least one structural motif in the plurality of structural motifs.

In certain embodiments, the step of decomposing a target structure into a plurality of structural motifs comprises identifying coupled residues in the target structure. Such coupled residues may be identified in the target structure, by finding position pairs capable of hosting amino acids that have an influence on each other via direct or indirect physical interactions, or through experimental evidence. In some embodiments, contact degree is used to identify coupled residues within a given structure.

For example, one method to determine whether a given pair of positions, i and j, are capable of forming contacts, is to first find all possible rotamers (of all amino acids) at both positions that do not clash with the backbone and then compute the weighted fraction of rotamer combinations at i and j that have closely approaching non-hydrogen atoms—i.e., contact degree.

An exemplary equation for computing contact degree is:

$$c ( i , j ) = \frac{\begin{matrix}{\sum }_{a ∈ AA} {\sum }_{b ∈ AA} {\sum }_{{r}_{i} ∈ {R}_{i} ( a )} {\sum }_{{r}_{j} ∈ {R}_{j} ( a )} {I}_{ij} ( {r}_{i} , {r}_{j} ) \\ Pr ( a ) Pr ( b ) p ( {r}_{i} ) p ( {r}_{j} )\end{matrix}}{\begin{matrix}{\sum }_{a ∈ AA} {\sum }_{b ∈ AA} {\sum }_{{r}_{i} ∈ {R}_{i} ( a )} {\sum }_{{r}_{j} ∈ {R}_{j} ( a )} \\ Pr ( a ) Pr ( b ) p ( {r}_{i} ) p ( {r}_{j} )\end{matrix}}$$

where Rᵢ(a) is a set of side-chain rotamers of amino acid a at position i (after discarding rotamers that clash with the backbone), Iᵢⱼ(rᵢ,rⱼ) is a binary variable indicating whether the two rotamers rᵢ and rⱼ would likely strongly influence each other's presence (have non-hydrogen atom pairs within 3 Å), Pr(a) is the frequency of amino acid a in the structural database, and p(rᵢ) is the probability of rotamer rᵢ. Rotamers and their probabilities can be taken from any backbone library. For example, Dunbrack and coworkers developed a backbone dependent library (Shapovalov M V & Dunbrack R L, Jr. (2011) A smoothed backbone-dependent rotamer library for proteins derived from adaptive kernel density estimates and regressions. Structure 19(6):844-858). By construction, the value c(i,j) varies between 0 and 1, with higher numbers corresponding to position pairs that are more poised to influence each other.

In certain embodiments, a contact-degree cutoff is used to identify which position pairs are to be considered coupled for the purposes of design calculations. For example, a contact-degree cutoff may be between about 0.01 to about 0.2, alternatively between about 0.01 and 0.1, or alternatively between about 0.01 and 0.05. In some such embodiments, the contact-degree cutoff is about 0.01. In other such embodiments, the contact-degree cutoff is about 0.05.

In certain embodiments, the step of decomposing a target structure into a plurality of structural motifs is guided by a graphical representation of (i) the target structure's coupled residues and/or (ii) the target structure's residue-backbone influences. Exemplary graphs, G and B, are shown in FIG. 4. In graph G, nodes represent residues and edges signify coupling, with edge weights optionally indicating the strength of coupling. In graph B, nodes represent residues and a directed edge a→b signifies that the backbone of b can influence the amino acid choice at a.

In certain embodiments, a sub-graph derived from the graphical representation of (i) the target structure's coupled residues and/or (ii) the target structure's residue-backbone influences identifies a structural motif. In some such embodiments, each structural motif in the plurality of structural motifs is formed around a set of one or more residues that represent a connected sub-graph of the graphical representation of coupled residues.

In certain embodiments, a secondary structural motif is defined around a given residue i to include residues (i−n) through (i+n), where n is a controllable parameter—we call this the singleton motif of i. For example, n may be between 1 and 10, such as 1, 2, 3, 4, 5, 6, 7, 8, 9, or 10. In some such embodiments, n is 1. In other such embodiments, n is 2.

In certain embodiments, a tertiary or quaternary structural motif is defined around a given residue, i, or more preferably, around the local backbone of residue i (e.g., (i−n) through (i+n), where i is a given position and n is a controllable parameter). For example, the process of identifying a structural motif may include residue i in isolation (e.g., a one-node subgraph) and consideration of some or all nodes to which residue i has directed edges (referring to Graph B, such a set may be called β(i)).

In certain embodiments, a structural motif is defined for each edge in the graphical representation of the target structure's coupled residues (e.g., Graph G). In some such embodiments, the structural motifs comprise each residue of in the pair as well as the associated singleton motifs.

In at least one aspect, this disclosure provides a method for computational protein design, the method comprising identifying, in a structural database, a plurality of structural matches for each of the plurality of structural motifs.

In certain embodiments, the structural database is the Protein Data Bank (PDB). In other such embodiments, the structural database is a specialized database containing, for example, only certain proteins, such as transmembrane proteins.

In some such embodiments, a quality filter is applied to the structural database. For example, a quality filter may assure that only high-quality structural data are available for searching. An exemplary quality filter only makes available entries solved by X-ray crystallography to a specified resolution, such as 2.6 Å or better. In some such embodiments, a redundancy filter is applied to the structural database. For example, a redundancy filter may remove unnecessary repetition to save computational time in querying the database. An exemplary redundancy filter removes overly redundant biological units, such as those having a specified sequence (%) identity to an already included biological unit. The specified sequence (%) identity may be, for example, >30%, >40%, >50%, >60%, >70%, >80%, or >90%.

In certain embodiments, the plurality of structural matches is obtained by querying the structural database. An exemplary search engine, MASTER, for querying structural databases is described in Zhou J & Grigoryan G (2014) Rapid search for tertiary fragments reveals protein sequence-structure relationships. Protein Science 24(4):508-524. In certain embodiments, the query encompasses backbone sub-structures from the database that align onto the backbone of the structural motif with low root-mean-square-deviation (RMSD). In some such embodiments, hydrogen atoms are excluded when calculating RMSD. In some such embodiments, search results are ordered by increasing RMSD.

In certain embodiments, the plurality of structural matches includes structural matches having an RMSD below a certain threshold. An exemplary size- and complexity-dependent RMSD cutoff function is:

$${RMSD}_{cut} = {σ}_{m} \sqrt{d / N}$$

$$d = N ( 1 - \frac{2}{N ( N - 1 )} {\sum }_{k} {\sum }_{i = 1}^{{n}_{k}} {\sum }_{j = i + 1}^{{n}_{k}} {e}^{[ ( i - j ) / L ]} )$$

where d is the effective number of degrees of freedom for the motif, nₖ is the length of the k-th contiguous segment of the motif, N is the total length of the motif (i.e., N=Σₖnₖ), L is correlation length—a parameter describing the extent of spatial correlation between residues in the same polypeptide chain, and σₘ is a plateau parameter. In certain embodiments, L is about 20 and σₘ is about 1.0 Å.

In certain embodiments, the plurality of structural matches includes N matches where N can be chosen based on the desired sample size necessary for subsequent pseudo-energy calculations. For example, N may be at least 100, at least 200, at least 300, at least 400, at least 500, at least 1000, at least 1500, or at least 2000. In some such embodiments, Nis 200. In some such embodiments, Nis 1000.

In certain embodiments, structural matches are screened for redundancy. In some such embodiments, structural matches are screened for sequence redundancy. In some such embodiments, structural matches are screened for structural redundancy.

For example, screening for sequence redundancy may comprise considering local sequence windows around each disjoint segment in match m and comparing these to the corresponding local sequence fragments from each of the previously obtained matches, μ, by aligning them via Needleman-Wunsch algorithm and the BLOSUM62 matrix. Local sequence windows can be defined as the segment of interest with 15 preceding and 15 succeeding residues, in the structure from which m originated. In some such embodiments, match m can be considered redundant with respect to match μ if any local sequence window alignment has a p-value less than about 10⁻³, alternatively less than about 10⁻⁴, alternatively less than about 10⁻⁵, or alternatively less than about 10⁻⁶. Alignment p-values may be computed based on alignment scores and indicate the probability that an alignment between sequences of the same length (chosen with database amino-acid frequencies) scores as well or better.

As another example, screening for structural redundancy may comprise identifying all residues in the structure from which match m originated that are coupled to any of the residues aligning to the corresponding query, Nₘ^{near}, and comparing match m to each of the previously obtained matches, μ, by calculating how many of its neighboring residues align well onto a neighboring residue of μ (defined as having a backbone RMSD below a specified threshold) in the orientation when both m and μ are optimally aligned to the query motif. In this context, an exemplary function for computing structural environment similarity between match m and previously obtained match μ is:

S_{m,μ}=N_{m,μ}^{near}/(0.5·[Nₘ^{near}+N_{μ}^{near}]+1)

In some such embodiments, match m can be considered redundant with respect to match ρ if S_{m,u} is above a specified cutoff. For example, the specified cutoff may be at least 0.1, at least 0.2, or at least 0.3. In some such embodiments, the specified cutoff is 0.2.

A2. Computation of Pseudo-Energetic Contributions

In at least one aspect, this disclosure provides a method for deducing a value for at least one non-local energetic contribution to a sequence-structure relationship for each of a plurality of structural matches to a tertiary or quaternary structural motif.

In certain embodiments, the at least one non-local energetic contribution is from a contiguous stretch of backbone around a single design position within one of the plurality of structural motifs (i.e., an own-backbone contribution). In certain embodiments, the at least one non-local energetic contribution is from a backbone in spatial but not sequence proximity to a single design position within one of the plurality of structural motifs (i.e., a near-backbone contribution). In certain embodiments, the at least one non-local energetic contribution is from a pair of coupled residues within one of the plurality of structural motifs (i.e., a pair contribution). In certain embodiments, the value for the at least one non-local energetic contribution is computed on-the-fly, while performing design calculations, by analyzing the structural motifs and their structural matches.

In certain embodiments, the method further comprises acquiring a value for at least one local energetic contribution to a sequence-structure relationship using each of the plurality of structural matches. In certain embodiments, the at least one local energetic contribution is from a backbone angle for a single design position within one of the plurality of structural motifs. In some such embodiments, the backbone angle is a phi, psi, or omega angle. In certain embodiments, the at least one local energetic contribution is from a burial state of a single design position within one of the plurality of structural motifs. In certain embodiments, the value for the at least one local energetic contribution is pre-computed based on the database.

In certain embodiments, the method comprises sequentially deducing a set of values for energetic contributions to a sequence-structure relationship using each of the plurality of structural matches according to a hierarchy of energetic contributions, the hierarchy comprising at least two of:
i. at least one local energetic contribution for a single design position within one of the plurality of structural motifs;
ii. a contiguous stretch of backbone around the single design position;
iii. a backbone in spatial but not sequence proximity to the single design position;
iv. a pair of coupled residues comprising the single design position; and
v. a triplet of residues comprising the single design position.

A2A. Backbone Angles

In certain embodiments, the method comprises deducing a value for at least one local energetic contribution. In some such embodiments, the local pseudo-energetic contribution describes the propensity of different amino acids for backbone φ (phi) and ψ (psi) dihedral angles. In some such embodiments, the pseudo-energetic contribution describing the propensity of different amino acids for backbone φ and dihedral angles is the first in a hierarchy of energetic contributions.

In certain embodiments, the pseudo-energetic contribution from the φ and ψ backbone angles is deduced by splitting the Φ/ψ phase-space into bins (e.g., bins of 10°×10°) and assigning each residue in a structural database into a corresponding bin based on its φ- and ψ-angle values. An exemplary function for computing a value for the pseudo-potential for amino acid a associated with backbone dihedrals bin Bᵢ^{φψ} is:

Eφψ(a|Bᵢ^{φψ})=−ln(f(a,Bᵢ^{φψ}))

where f(a,Bᵢ^{φψ}) is the frequency with which amino acid a is found in this bin within proteins in the structural database:

$$f ( a , {B}_{i}^{ϕψ} ) = N ( a , {B}_{i}^{ϕψ} ) / {\sum }_{aa = 1}^{20} N ( aa , {B}_{i}^{ϕψ} )$$

N(aa,Bᵢ^{φψ}) being the number of times amino acid aa is found in bin Bᵢ^{φψ}.

In certain embodiments, the method comprises deducing a value for at least one local energetic contribution. In some such embodiments, the local pseudo-energetic contribution describes the preference of amino acids for different backbone ω (omega) dihedral angles. In some such embodiments, the pseudo-energetic contribution describing the preference of amino acids for different backbone ω dihedral angles is the second in a hierarchy of energetic contributions (e.g., considered only after considering the local pseudo-energetic contribution describes the propensity of different amino acids for backbone φ (phi) and ψ (psi) dihedral angles).

In certain embodiments, the pseudo-energetic contribution from the ω dihedral angles is deduced by splitting the ω phase-space into bins and assigning each residue in a structural database into a corresponding bin based on its ω-angle values. Because the ω angle is defined around the peptide bond, which has partial double-bond character, ω angles are typically planar, with values close to 180° most common (trans peptide bonds), but values around 0° also occurring (cis peptide bonds), generally (though not exclusively) with Pro or Gly amino acids. Thus, in some such embodiments, the method comprises a non-uniform binning of ω angles, where bin widths are at least 1°, but as large as needed to have a sufficient number of structural database residues in each bin.

An exemplary function for computing a value for the pseudo-potential for amino acid a associated with ω-angle bin Bᵢ^{ω} is:

$${E}^{ω} ( a \vert {B}_{i}^{ω} ) = - ln ( \frac{N ( a , {B}_{i}^{ω} ) + {ɛ}_{ω}}{{N}_{e} ( a , {B}_{i}^{ω} ) + {ɛ}_{ω}} )$$

where N(a,Bᵢ^{ω}) is the number of times amino acid a is found in bin Bᵢ^{ω}, and Nₑ(a,Bᵢ^{ω}) is the number of times a is expected to be found in the bin, based on the pseudo-energetic contributions already known—for example, the φ/ψ energy, and ε_{ω} acting as a pseudo-count, preventing excessive statistical noise from poorly populated bins. In some such embodiments, ε_{ω} is 1.

An exemplary function for Nₑ(a,Bᵢ^{ω}) is:

$${N}_{e} ( a , {B}_{i}^{ω} ) = {\sum }_{k ∈ {B}_{i}^{ω}} \frac{exp ( - {E}^{ϕψ} ( a \vert {B}^{ϕ ψ} ( k ) ) )}{{\sum }_{aa ∈ AA} exp ( - {E}^{ϕ ψ} ( aa \vert {B}^{ϕ ψ} ( k ) ) )}$$

where the outer sum is over all native residues falling into ω bin Bᵢ^{ω}, the inner sum is over all natural amino acids, denoted by set AA, and B^{φψ}(k) is the φ/ψ bin into which residue k falls. The inner fraction represents the expected probability of observing a (over all possible amino acids) in the φ/ψ environment of each residue in the bin. The correction by expectation in the equation above assures that E^{ω} acts only as a corrector over E^{φψ}, explaining only what is not already explained in the data.

A2B. Burial State

In certain embodiments, the method comprises deducing a value for at least one local energetic contribution. In some such embodiments, the local pseudo-energetic contribution is from a general environment (i.e., burial state) of a residue. In some such embodiments, the pseudo-energetic contribution from the burial state of a residue is a subsequent contribution in a hierarchy of energetic contributions (e.g., considered only after considering the local pseudo-energetic contribution describing the propensity of different amino acids for backbone φ and ψ dihedral angles and the local pseudo-energetic contribution describing the preference of amino acids for different backbone ω dihedral angles).

In certain embodiments, the pseudo-energetic contribution from the burial state is deduced by computing an environmental descriptor, e, for all residues in the structural database and binning the residues according to e. To capture the contribution from the burial state of a residue as a single-body (self) contribution, the environmental descriptor may be a sequence-independent environmental descriptor.

An exemplary function for computing a value for the pseudo-potential for amino acid a associated with environment bin Bᵢ^{e} is:

$${E}^{e} ( a \vert {B}_{i}^{e} ) = - ln ( \frac{N ( a , {B}_{i}^{e} ) + {ɛ}_{e}}{{N}_{e} ( a , {B}_{i}^{e} ) + {ɛ}_{e}} )$$

where N(a,Bᵢ^{e}) is the number of times amino acid a is found in bin Bᵢ^{e}, and Nₑ(a,Bᵢ^{e}) is the number of times a is expected to be found in the bin, based on the pseudo-energetic contributions already known—for example, the φ/ψ energy and ω energy, and εₑ acting as a pseudo-count, preventing excessive statistical noise from poorly populated bins. In some such embodiments, εₑ is 1.

An exemplary function for Nₑ(a,Bᵢ^{e}) is:

$${N}_{e} ( a , {B}_{i}^{e} ) = {\sum }_{k ∈ {B}_{i}^{e}} \frac{exp ( - {E}^{ϕψ} ( a \vert {B}^{ϕ ψ} ( k ) ) - {E}^{ω} ( a \vert {B}^{ω} ( k ) ) )}{{\sum }_{aa ∈ AA} exp ( - {E}^{ϕ ψ} ( aa \vert {B}^{ϕ ψ} ( k ) ) - {E}^{ω} ( aa \vert {B}^{ω} ( k ) ) )}$$

where the outer sum is over all native residues assigned to the environment bin Bᵢ^{e}, and B^{ω}(k) is the ω bin into which residue k maps. The correction by expectation in the equation above assures that E^{e} acts only as a corrector over what is already explained by pseudo-energetic contributions considered earlier in the hierarchy (e.g., E^{φψ} and/or E^{ω}).

A variety of sequence-independent environmental descriptors, e, may be used. In one embodiment, the sequence-independent environmental descriptor may be “residue freedom”, which considers all possible rotamers of all natural amino acids at a given position and its surroundings to determine the extent to which the volume around the residue would tend to be unoccupied and available to its rotamers. An exemplary function for freedom for a given residue i, F(i), is:

$$F ( i ) = \sqrt{\frac{{V}_{i , 0.5}^{2} + {V}_{i , 2}^{2}}{2}} where$$

$${V}_{i , τ} = \frac{{\sum }_{a ∈ AA} {\sum }_{{r}_{i} ∈ {R}_{i} ( a )} I ( {p}_{c} ( {r}_{i} ) < τ )}{{\sum }_{aa ∈ AA} \vert {R}_{i} ( a ) \vert} , and$$

$${p}_{c} ( {r}_{i} ) = {\sum }_{j \neq i} {\sum }_{b ∈ AA} {\sum }_{{r}_{j} ∈ {R}_{j} ( b )} {I}_{ij} ( {r}_{i} , {r}_{j} ) Pr ( b ) p ( {r}_{j} )$$

where Rᵢ(a) is a set of side-chain rotamers of amino acid a at position i (after discarding rotamers that clash with the backbone), Iᵢⱼ(rᵢ,rⱼ) is a binary variable indicating whether the two rotamers rᵢ and rⱼ would likely strongly influence each other's presence (have non-hydrogen atom pairs within 3 Å), Pr(a) is the frequency of amino acid a in the structural database, and p(rᵢ) is the probability of rotamer rᵢ; and where p_{c}(rᵢ) is the “collision probability mass” or rotamer rᵢ—i.e., how likely it is to clash with rotamers at other positions.

A2C. Own-Backbone

In certain embodiments, the method comprises deducing a value for at least one non-local pseudo-energetic contribution. In some such embodiments, the non-local pseudo-energetic contribution is from a contiguous stretch of backbone around a single design position at a given position (i.e., an own-backbone contribution). In some such embodiments, the own-backbone contribution is a subsequent contribution in a hierarchy of energetic contributions (e.g., considered only after considering one or more local pseudo-energetic contributions).

In certain embodiments, the own-backbone contribution captures how the local contiguous stretch of backbone around position p modulates its amino-acid preferences, beyond what is already captured by φ/ψ, ω, and burial state preferences.

In certain embodiments, the own-backbone contribution is deduced by excising from the target structure a structural motif comprising position p and its surrounding contiguous backbone fragment, Tₚ, and identifying structural matches to Tₚ in the structural database. The set of structural matches is referred to as Mₚ.

An exemplary function for computing a value for the own-backbone contribution for amino acid a in position p:

$${E}_{p}^{o} ( a \vert D ) = - ln ( \frac{N ( a , {M}_{p} ) + {ɛ}_{o}}{{N}_{e} ( a , {M}_{p} ) + {ɛ}_{o}} )$$

where N(a,Mₚ) is the number of times amino acid a is observed in the position corresponding to p within the set of structural matches Mₚ and Nₑ(a,Mₚ) is the number of times a is expected to be in this position, based on the pseudo-energetic contributions already known—for example, the φ/ψ, ω, and/or environment energies—and εₒ acting as a pseudo-count. In some such embodiments, εₒ is 1.

An exemplary function for Nₑ(a,Mₚ) is:

$${N}_{e} ( a , {M}_{p} ) = {\sum }_{m ∈ {M}_{p}} \frac{exp ( - {E}^{ϕ ψ} ( a \vert {B}^{ϕψ} ( {m}_{p} ) ) - {E}^{ω} ( a \vert {B}^{ω} ( {m}_{p} ) ) - {E}^{e} ( a \vert {B}^{e} ( {m}_{p} ) ) )}{{\sum }_{aa ∈ AA} exp ( \begin{matrix}- {E}^{ϕ ψ} ( aa \vert {B}^{ϕ ψ} ( {m}_{p} ) ) - {E}^{ω} ( aa \vert {B}^{ω} ( {m}_{p} ) ) - \\ {E}^{e} ( aa \vert {B}^{e} ( {m}_{p} ) )\end{matrix} )}$$

where the outer sum is over matches in Mₚ, mₚ is the residue in match m that aligns with position p in Tₚ, and Bₑ(mₚ) is the environment bin to which mₚ belongs, based on its surroundings in the structure from which match m originates.

A2D. Near-Backbone

In certain embodiments, the method comprises deducing a value for at least one non-local pseudo-energetic contribution. In some such embodiments, the non-local pseudo-energetic contribution is from a backbone in spatial but not sequence proximity to a single design position at a given position (i.e., a near-backbone contribution). In some such embodiments, the near-backbone contribution is a subsequent contribution in a hierarchy of energetic contributions (e.g., considered only after considering one or more local pseudo-energetic contributions and the own-backbone contribution).

In certain embodiments, the near-backbone contribution captures any further modulation of amino acid preferences at position p brought about by the presence of backbone segments in close spatial but not sequence proximity to position p.

In certain embodiments, the near-backbone contribution is deduced by excising from the target structure a structural motif comprising position p, its surrounding contiguous backbone segment, and backbone segments in close spatial (but not sequence) proximity to p, T′_{p,t}, and identifying structural matches to T′_{p,t} in the structural database; subscript t indicates that multiple such structural motifs are possible. The set of structural matches is referred to as M′_{p,t}.

An exemplary function for computing a value for the near-backbone contribution for amino acid a in T′_{p,t}:

$${E}_{p , t}^{′} ( a \vert D ) = - ln ( \frac{N ( a , {M}_{p , t}^{′} ) + {ɛ}_{n}}{{N}_{e} ( a , {M}_{p , t}^{′} ) + {ɛ}_{n}} )$$

where N(a,M′_{p,t}) is the number of times amino acid a is observed in the position corresponding top within the set of structural matches M′_{p,t} and Nₑ(a,M′_{p,t}) is the number of times a is expected to be in this position, based on the pseudo-energetic contributions already known—for example, the φ/ψ, ω, environment, and/or own-backbone energies—and εₙ acting as a pseudo-count. In some such embodiments, εₙ is 1.

An exemplary function for Nₑ(a,M′_{p,t}) is:

$${N}_{e} ( a , {M}_{p , t}^{′} ) = {\sum }_{m ∈ {M}_{p , t}^{′}} \frac{exp ( \begin{matrix}- {E}^{ϕ ψ} ( a \vert {B}^{ϕψ} ( {m}_{p} ) ) - \\ {E}^{ω} ( a \vert {B}^{ω} ( {m}_{p} ) ) - {E}^{e} ( a \vert {B}^{e} ( {m}_{p} ) ) - {E}_{p}^{o} ( a \vert m )\end{matrix} )}{{\sum }_{aa ∈ AA} exp ( \begin{matrix}- {E}^{ϕ ψ} ( aa \vert {B}^{ϕ ψ} ( {m}_{p} ) ) - {E}^{ω} ( aa \vert {B}^{ω} ( {m}_{p} ) ) - \\ {E}^{e} ( aa \vert {B}^{e} ( {m}_{p} ) ) - {E}_{p}^{o} ( aa \vert m )\end{matrix} )}$$

where the outer sum is over matches in M′_{p,t}, and Eₚ^{o} (a|m) represents the own-backbone pseudo-energy for amino acid a in residue mₚ, based on the structure from which match m originates.

A2E. Pair

In certain embodiments, the method comprises deducing a value for at least one non-local pseudo-energetic contribution. In some such embodiments, the non-local pseudo-energetic contribution is from a pair of coupled residues, (p, q) in the target structure (i.e., a pair pseudo-energy contribution). In some such embodiments, the pair contribution is a subsequent contribution in a hierarchy of energetic contributions (e.g., considered only after considering one or more local pseudo-energetic contributions, an own-backbone contribution, and/or a near-backbone contribution).

In certain embodiments, the pair contribution is deduced by excising from the target structure a structural motif comprising positions p and q, T″_{p,q}, and identifying structural matches to T″_{p,q} in the structural database. The set of structural matches is referred to as M″_{p,q}.

An exemplary function for computing a value for the pair contribution for amino acids a and b in positions p and q, respectively, in T″_{p,q}:

$${E}_{p , q}^{″} ( a , b \vert D ) = - ln ( \frac{N ( a , b , {M}_{p , q}^{″} ) + {ɛ}_{p}}{{N}_{e} ( a , b , {M}_{p , q}^{″} ) + {ɛ}_{p}} )$$

where N(a,b,M″_{p,q}) is the number of times amino acids a and b are observed in the positions corresponding top and q within the set of structural matches M″_{p,q} and Nₑ(a,b,M″_{p,q}) is the number of times (a, b) pair is expected to be in these positions, based on the pseudo-energetic contributions already known—for example, the φ/ψ, ω, environment, own-backbone, and/or near-backbone energies—and ϑₚ acting as a pseudo-count. In some such embodiments, εₚ is 1.

An exemplary function for Nₑ(a,b,M″_{p,q}) is:

$${N}_{e} ( a , b , {M}_{p , q}^{″} ) = {\sum }_{m ∈ {M}_{p , q}^{″}} \frac{exp ( - {E}_{lo} ( a \vert {m}_{p} ) - {Δ}_{p} ( a , {M}_{p , q}^{″} ) )}{{\sum }_{aa ∈ AA} exp ( - {E}_{lo} ( aa \vert {m}_{p} ) - {Δ}_{p} ( a , {M}_{p , q}^{″} ) )} \times \frac{exp ( - {E}_{lo} ( b \vert {m}_{q} ) - {Δ}_{q} ( b , {M}_{p , q}^{″} ) )}{{\sum }_{aa ∈ AA} exp ( - {E}_{lo} ( aa \vert {m}_{q} ) - {Δ}_{q} ( a , {M}_{p , q}^{″} ) )}$$

where, for brevity, Eₗₒ(a|mₚ) denotes the total pseudo-energy from all lower contributions considered thus far, associated with amino acid a in the position aligned with position p of match m:

$${E}_{lo} ( a \vert {m}_{p} ) = {E}^{ϕ ψ} ( a \vert {B}^{ϕ ψ} ( {m}_{p} ) ) + {E}^{ω} ( a \vert {B}^{ω} ( {m}_{p} ) ) + {E}^{e} ( a \vert {B}^{e} ( {m}_{p} ) ) + {E}_{p}^{o} ( a \vert m ) + {\sum }_{t} {E}_{p , t}^{′} ( a \vert m )$$

and Δₚ(a, M″_{p,q}) is an optional adjustment energy that can be included to preserve the marginal amino acid distributions at individual coupled positions of the structural motif.

A2F. Triplet

In certain embodiments, the method comprises deducing a value for at least one non-local pseudo-energetic contribution. In some such embodiments, the non-local pseudo-energetic contribution is from a triplet of residues, (p, q, r) in the target structure (i.e., a triplet pseudo-energy contribution). In some such embodiments, the triplet contribution is a subsequent contribution in a hierarchy of energetic contributions (e.g., considered only after considering one or more local pseudo-energetic contributions, an own-backbone contribution, a near-backbone contribution, and/or a pair contribution).

In certain embodiments, the triplet contribution is deduced by excising from the target structure a structural motif comprising positions p, q, and r, T′″_{p,q,r}, and identifying structural matches to T′″_{p,q,r} in the structural database. The set of structural matches is referred to as M′″_{p,q,r}.

An exemplary function for computing a value for the pair contribution for amino acids a, b, and c in positions p, q, and r, respectively, in T′″_{p,q,r}:

$${E}_{p , q , r}^{′′′} ( a , b , c \vert D ) = - ln ( \frac{N ( a , b , c , {M}_{p , q , r}^{′′′} ) + {ɛ}_{t}}{{N}_{e} ( a , b , c , {M}_{p , q , r}^{′′′} ) + {ɛ}_{t}} )$$

where N(a,b,c,M′″_{p,q,r}) is the number of times the triplet (a,b,c) is observed in positions corresponding to (p,q,r) within the set of structural matches M′″_{p,q,r} and Nₑ(a,b,c,M′″_{p,q,r}) is the number of times (a,b,c) triplet is expected to be in these positions, based on the pseudo-energetic contributions already known—for example, the φ/ψ, ω, environment, own-backbone, near-backbone, and/or pair energies—and εₜ acting as a pseudo-count. In some such embodiments, εₜ is 1.

An exemplary function for Nₑ(a,b,c,M′″_{p,q,r}) is:

$${N}_{e} ( a , b , c , {M}_{p , q , r}^{′′′} ) = {\sum }_{m ∈ {M}_{p , q}^{″}}^{ } \frac{exp ( \begin{matrix}- {E}_{lo} ( a , b , c \vert {m}_{p , q , r} ) - {Δ}_{p} ( a , {M}_{p , q , r}^{′′′} ) - \\ \begin{matrix}{Δ}_{q} ( a , {M}_{p , q , r}^{′′′} ) - {Δ}_{r} ( c , {M}_{p , q , r}^{′′′} ) - \\ {Δ}_{p , q} ( a , b , {M}_{p , q , r}^{′′′} ) - {Δ}_{p , r} ( a , c , {M}_{p , q , r}^{′′′} ) - {Δ}_{q , q} ( b , c , {M}_{p , q , r}^{′′′} )\end{matrix}\end{matrix} )}{{\sum }_{α , β , γ ∈ AA} exp ( \begin{matrix}- {E}_{lo} ( a , b , c \vert {m}_{p , q , r} ) - {Δ}_{p} ( a , {M}_{p , q , r}^{′′′} ) - \\ {Δ}_{r} ( c , {M}_{p , q , r}^{′′′} ) - {Δ}_{p , q} ( a , b , {M}_{p , q , r}^{′′′} ) - \\ {Δ}_{p , r} ( a , c , {M}_{p , q , r}^{′′′} ) - {Δ}_{q , q} ( b , c , {M}_{p , q , r}^{′′′} )\end{matrix} )}$$

where, for brevity, Eₗₒ(a, b, c|m_{p,q,r}) denotes the total pseudo-energy from all lower contributions considered thus far, associated with amino acid a in the position aligned with positions p, q, and r of match m:

$${E}_{lo} ( a , b , c \vert {m}_{p , q , γ} ) = {\sum }_{x = ( p , q , r )} [ {E}^{ϕψ} ( {aa}_{x} \vert {B}^{ϕ ψ} ( {m}_{x} ) ) + {E}^{ω} ( {aa}_{x} \vert {B}^{ω} ( {m}_{x} ) ) + {E}^{p} ( {aa}_{r} \vert {B}^{e} ( {m}_{x} ) ) + {E}_{x}^{o} ( {aa}_{x} \vert m ) + {\sum }_{e} {E}_{xx}^{′} ( {aa}_{x} \vert m ) ] + {{\sum }_{x , y = ( p , q , r )}}_{x < y} {E}_{x , y}^{″} ( {aa}_{x} , {aa}_{y} \vert m )$$

and Δ_{p,q}(a, b, M′″_{p,q,r}) is an optional adjustment energy that can be included to constrain the pairwise amino acid distributions at pairs of positions in T′″_{p,q,r}.

A3. Protein Optimization

In at least one aspect, this disclosure provides a method for determining an amino acid sequence or a library of amino acid sequences capable of folding into a binding partner of the target structure. A library of amino acid sequences may comprise a set of amino acids sequences having, for example, at most about 50%, alternatively at most about 60%, alternatively at most about 70%, alternatively at most about 80%, or alternatively at most about 90% sequence identity to each other. In certain embodiments, the set of amino acid sequences comprises variants of a core, generic sequence.

In certain embodiments, an optimization approach is used to determine the amino acid sequence or the library of amino acid sequences capable of folding into a binding partner of the target structure. For example, once all values for pseudo-energetic contributions are computed and, optionally, organized into a table of self, pair, and possibly higher-order pseudo-energetic contributions, a host of optimization approaches can be used to deduce the optimal amino acid sequence. In certain embodiments, an Integer Linear Programming (ILP) approach is used. The ILP approach allows for the introduction of constraints into the design problem (e.g., sequence symmetry constraints, or constraints on the number of charged/polar or hydrophobic residues, or limits on the residues mutated relative to some starting sequence). In certain embodiments, alternative optimization methods are used—for example, Self-Consistent Mean Field (SCMF) or Simulated Annealing Monte Carlo (MC). In certain embodiments, identification of an absolute global optimal sequence is not required; any close-to-optimal sequence is sufficient.

### B. PROTEIN EXPRESSION

In certain aspects, a product of the methods described herein is an amino acid sequence or a library or set of amino acid sequences, which are recommended for expression and further optimization using experimental in vitro and/or in vivo procedures.

In a further aspect, this disclosure provides a nucleic acid sequence encoding a computationally designed protein provided herein. Such nucleic acid sequences may further comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals.

In certain embodiments, the nucleic acid sequence is contained in a vector (e.g., a plasmid, cosmid, virus, bacteriophage or another vector conventionally used in genetic engineering). In some such embodiments, the vector comprises expression control elements allowing proper expression of the coding regions in suitable host cells. “Control elements” operably linked to the nucleic acid sequence encoding the computationally designed protein are further nucleic acid sequences capable of effecting the expression of the computationally designed protein. For example, a control element may include any of a variety of constitutive promoters, including but not limited to CMV, SV40, RSV, or actin, or inducible promotors, including but not limited to promoters driven by tetracycline or a steroid. The control elements need not be contiguous with the protein-encoding nucleic acid sequence, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, initiation signals, polyadenylation signals, termination signals, and ribosome binding sites. In certain embodiments, the vector comprises further genes such as marker genes which allow for the selection of the vector in a suitable host cell and under suitable conditions. Methods for construction of nucleic acid molecules, for construction of vectors comprising nucleic acid molecules, for introduction of vectors into appropriately chosen host cells, or for causing or achieving expression of nucleic acid molecules are well-known in the art.

In another aspect, this disclosure provides a host cell comprising a nucleic acid or vector as disclosed herein. The host cell can be either prokaryotic or eukaryotic. The host cell can be transiently or stably transfected. Such transfection of expression vectors into prokaryotic and eukaryotic cells can be accomplished via any technique known in the art, including but not limited to standard bacterial transformations, calcium phosphate co-precipitation, electroporation, or liposome mediated-, DEAE dextran mediated-, polycationic mediated-, or viral mediated transfection.

In a further aspect, this disclosure provides a method for producing a computationally designed protein. The method comprises the steps of (a) culturing a host cell comprising a nucleic acid sequence encoding the protein under conditions conducive to the expression of the protein, and (b) optionally, recovering the expressed protein. Hence, in certain embodiments, the method for producing a computationally designed protein comprises: designing and selecting at least one amino acid sequence; expressing the amino acid sequence in an expression system, thereby producing the computationally designed protein. In certain embodiments, the amino acid sequence is a protein that is capable of folding into a binding partner of a target structure.

In some such embodiments, the method comprises generating, in silico, at least one candidate amino acid sequence; introducing a nucleic acid sequence encoding the candidate amino acid sequence into a host cell; and expressing the candidate amino acid sequence. In some such embodiments, the method further comprises determining whether the candidate amino acid sequence folds into a binding partner of the target structure. Such a determination can be made by known methods to assess protein binding, including biochemical and/or biophysical methods.

In certain embodiments, the computationally designed protein is an enzyme, antibody, receptor, ligand, transport protein, hormone, growth factor, and a fragment thereof. In some such embodiments, the antibody is a human antibody. In some such embodiments, the computationally designed protein is a single chain antibody, e.g., single chain Fv. In some such embodiments, the computationally designed protein is an antigen-binding antibody fragment such as a Fab or Fab′ fragment.

### C. DEFINITIONS

As used herein, “contact degree” refers to the opportunity that a given pair of positions, i and j, have to establish contacts. Contact degree can be used to identify “coupled residues.”

As used herein, “coupled residues” refers to a pair of amino acid residues in, for example a target structure, where the amino acid identity of one residue depends on the amino acid identity of the other residue in the pair.

In this disclosure, the use of the disjunctive is intended to include the conjunctive. The use of definite or indefinite articles is not intended to indicate cardinality. In particular, a reference to “the” object or “a” and “an” object is intended to denote also one of a possible plurality of such objects. Further, the conjunction “or” may be used to convey features that are simultaneously present instead of mutually exclusive alternatives. In other words, the conjunction “or” should be understood to include “and/or”. The terms “includes,” “including,” and “include” are inclusive and have the same scope as “comprises,” “comprising,” and “comprise” respectively.

The above-described embodiments, and particularly any “preferred” embodiments, are possible examples of implementations and merely set forth for a clear understanding of the principles of the invention. Many variations and modifications may be made to the above-described embodiment(s) without substantially departing from the spirit and principles of the techniques described herein. All modifications are intended to be included herein within the scope of this disclosure and protected by the following claims.

### D. EXAMPLES

The following examples are merely illustrative, and not limiting to this disclosure in any way.

### Example 1: Surface Redesign (Resurfacing)

Protein surfaces—i.e., the set of residues exposed to solvent—are important in determining a multitude of biophysical properties, including solubility, immunogenicity, self-association, propensity for aggregation, as well as stability and fold specificity. It is, therefore, sometimes useful to redesign just the surface of a given protein, so as to modulate one or more of these properties, while preserving its overall structure and function. This Example describes the task of redesigning the surface (resurfacing) of a Red Fluorescent Protein (RFP). RFPs are proteins that naturally fluoresce, with the emission spectrum concentrated around the red portion of the visible spectrum (˜600 nm). Like other fluorescent proteins (FPs), RPFs are of high utility as biological imaging tags and in optical experiments [1]. It may therefore be useful to modulate the surface residues of an RFP depending on the environment (or cell type) in which it has to function (often at high concentration).

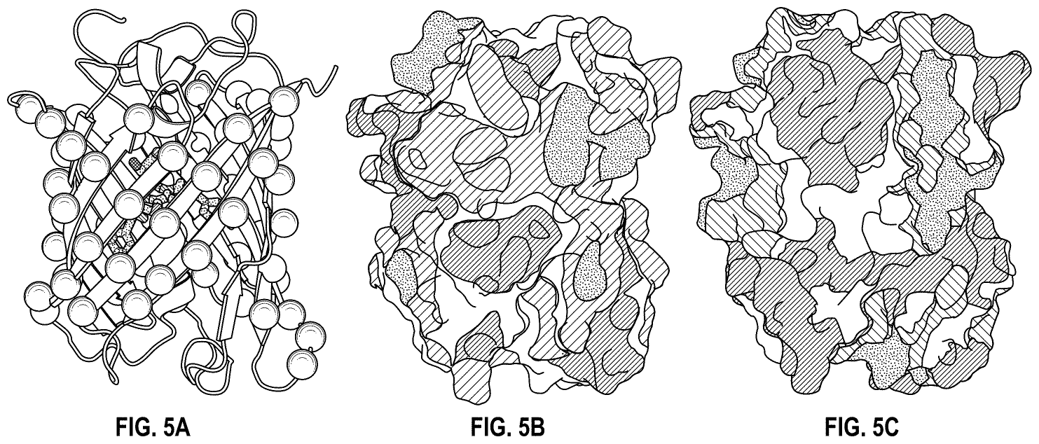

The crystal structure of RFP mCherry (PDB code 2H5Q [2]) was used as the design template. A total of 64 positions in the structure were manually chosen as being on the surface (roughly corresponding to positions with freedom values above 0.42); these are shown as spheres in FIG. 5 (left panel). Following this, an exemplary TERM-based method described herein was used to compute a statistical energy table corresponding to all of the surface positions varying among the twenty natural amino acids, with the remaining positions fixed to their identities in the PDB entry 2H5Q. The resulting energy table, therefore, described a sequence space of 20⁶⁴≈2*10⁸³ sequences. Integer linear programing was used to optimize over this space, finding the single sequence with the lowest total statistical energy score. The resulting sequence, compared to the starting sequence of mCherry, is shown in Table 1. The in-vacuo surface electrostatic potential of the original mCherry structure and the resulting design model structure are compared in FIG. 5 (middle and right panels); clearly, the designed sequence represents a significant perturbation to the electrostatics and the shape of the surface. In fact, a total of 48 out of 64 variable positions are changed in the design.

TABLE 1
TERM-based designed sequence differs significantly from
the original wild-type mCherry sequence.

| | |
|---|---|
| mCherry | MVSKGEEDNM AIIKEFMRFK VHMEGSVNGH EFEIEGEGEG RPYEGTQTAK |
| design | MVSKGEEDNM AIIKEFMTFE VEMEGTVNGH PFRIRGSGGG DPYEGTQTAR |
| mCherry | LKVTKGGPLP FAWDILSPQF MYGSKAYVKH PADIPDYLKL SFPEGFKWER |
| design | LEVVEGGPLP FAWDILSPQF MYGSKAYVKH PADIPDYLKL SFPEGFTWTR |
| mCherry | VMNFEDGGVV TVTQDSSLQD GEFIYKVKLR GTNFPSDGPV MQKKTMGWEA |
| design | TMEFEDGGTV KVTQTSTLKD GKFHYKVKLT GSNFPSDGPV MQKKTMGWEA |
| mCherry | SSERMYPEDG ALKGEIKQRL KLKDGGHYDA EVKTTYKAKK PVQLPGAYNV |
| design | STERMRPKDG KLEGEIDQEL RLKDGGYYRA RVRTTYKAKK PVQLPGAYTV |
| mCherry | NIKLDITSHN EDYTIVEQYE RAEGRHSTGG MDELYK (SEQ ID NO. 1) |
| design | RIRLEITSHN EDYTEVEQTE TAKGEHSTGG MDELYK (SEQ ID NO. 2) |

Positions marked as variable in design are underlined, and those mutated in the designed sequence additionally marked in bold.

To validate the design, the sequence was cloned into E. coli, followed by expression and purification using standard molecular biological and biophysical techniques.

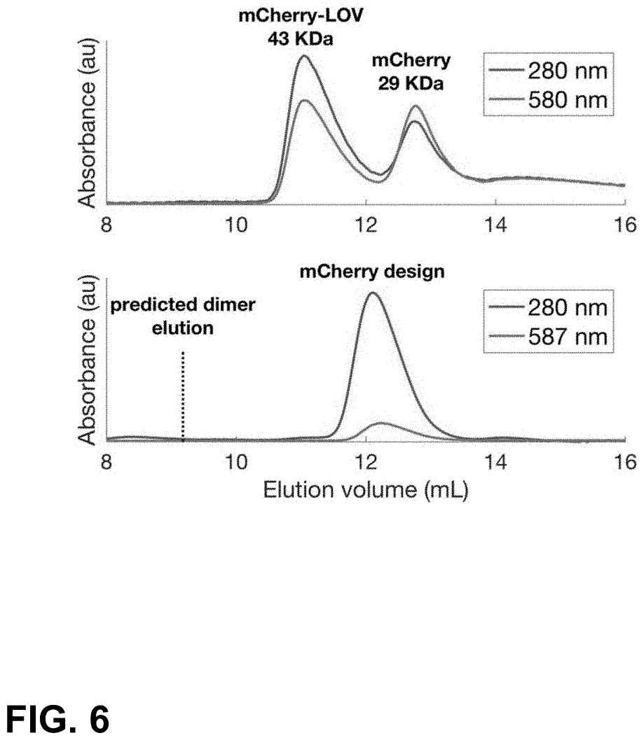

Fast Protein Liquid Chromatography (FPLC) showed the protein to be monomeric in solution (at concentration of at least 10 μM), just as the native mCherry (see FIG. 6).

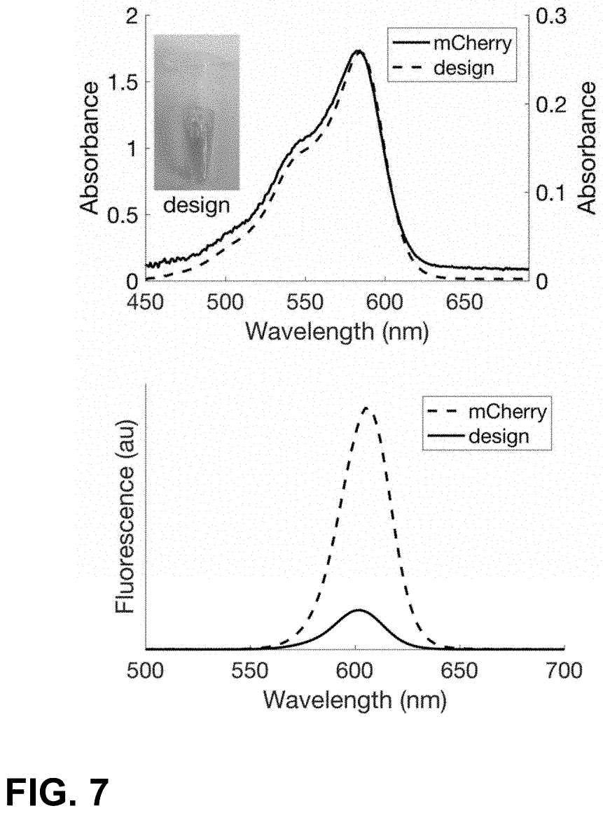

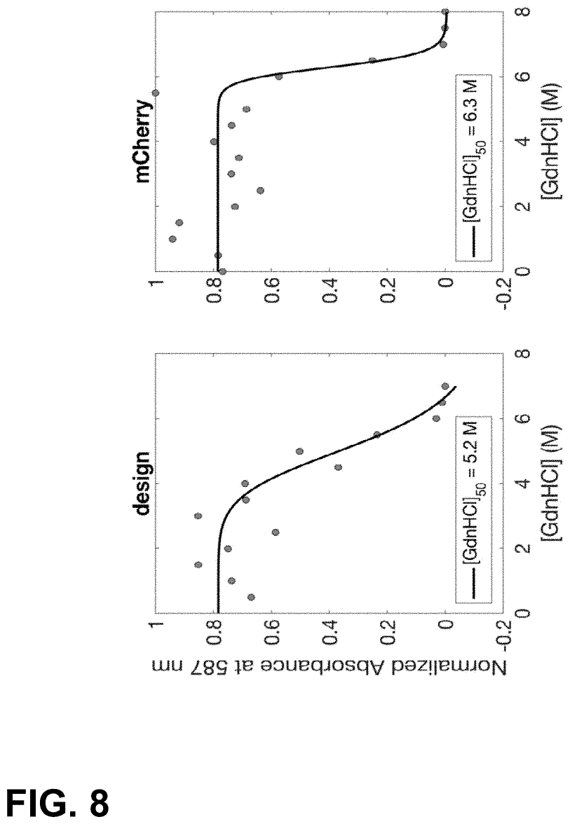

Despite harboring 48 mutations and despite the fact that preservation of optical properties was not a design constraint (only preservation of structure was), the design still exhibited the pink color characteristic of the original protein (see FIG. 7, top). Further, the designed protein was still fluorescent, with an emission spectrum exhibiting nearly identical shape to that of mCherry (see FIG. 7, bottom). Finally, chemical denaturation by guanidinium hydrochloride (GuHCl) revealed that the protein's structure protected its chromophore approximately as well as the original mCherry—a hyper-stable, highly engineered protein in its own right (FIG. 8). Thus, by all measures, the designed protein, which differs from the original mCherry in 48 positions, preserved the starting structure and even function. The ability to generate such diversity can be easily exploited to quickly engineer variants of RFP or other proteins that possess a range of desired properties.

### Example 2: Resurfacing for the Solubilization of Membrane Proteins

Notably, the resurfacing approach can be used to redesign membrane proteins for solubility in aqueous solution (5). Water-soluble proteins are much easier to express, purify, and manipulate than transmembrane (TM) proteins, making them easier subjects for therapeutic targeting. Thus, the ability to produce water-soluble analogues of membrane proteins could simplify considerably the process of identifying drugs and antibodies against key biomedically-relevant targets, such as G protein-coupled receptors (GPCRs).

The use of TERM-based design for this purpose includes identifying lipid-facing positions on the surface of a TM protein structure, which would become solvent-exposed upon solubilization in water, and redesigning them via the standard procedure as employed in Example 1 above.

The specific choices of amino-acid combinations between interacting surface positions arose as a result of observing and “learning” sequence statistics in similar structural environments of known water-soluble protein structures, which can be a part of the design procedures disclosed herein.

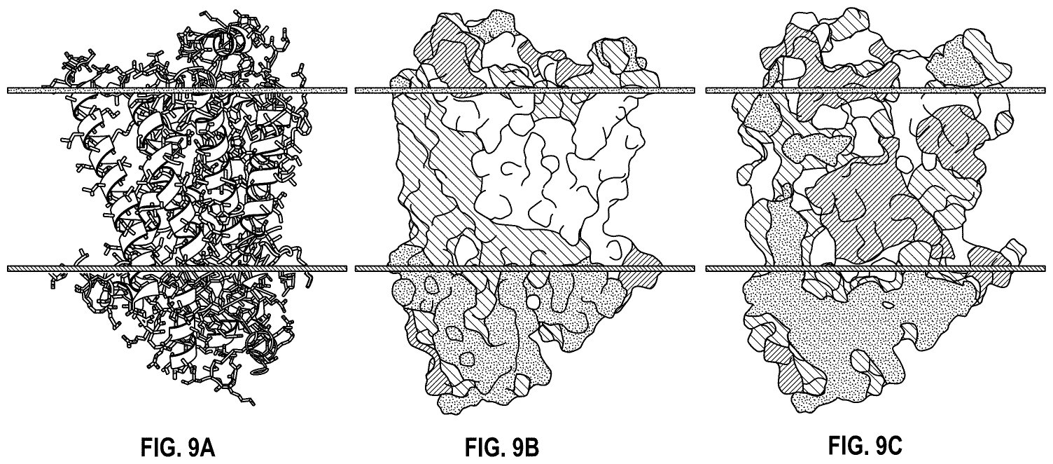

FIG. 9 shows the result of applying this process to the crystal structure of GPCR beta-1 adrenergic receptor (PDB code 4BVN, see left panel). Comparing the middle and right panels of FIG. 9, it is evident that the design process transformed the surface of the protein from a mostly hydrophobic one, ideal for interacting with the lipid bilayer, to a hydrophilic one well suited for interacting with water. Thus, the methods described herein are useful to resurface a protein, such as a GPCR, for water solubility.

### Example 3: Statistical Energy Scores Computed by TERM-Based Methods Indicate Design Quality

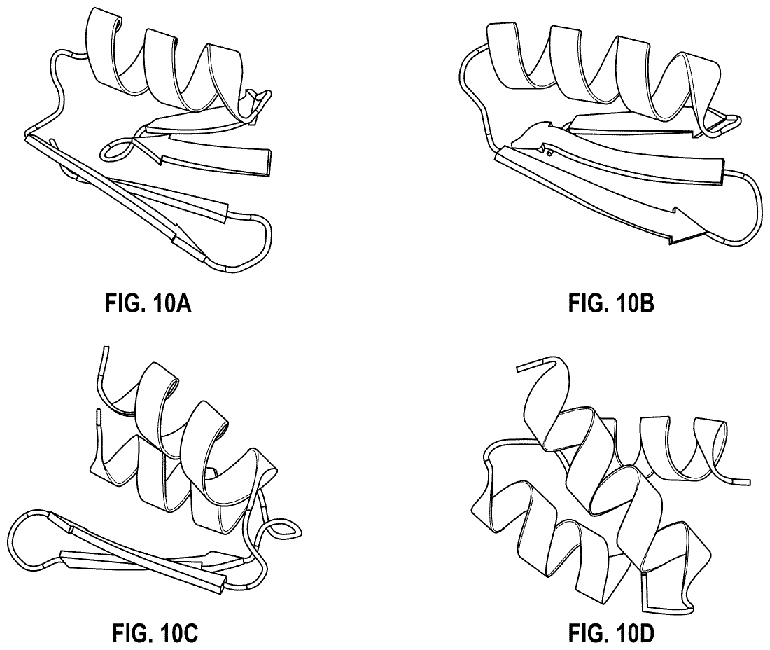

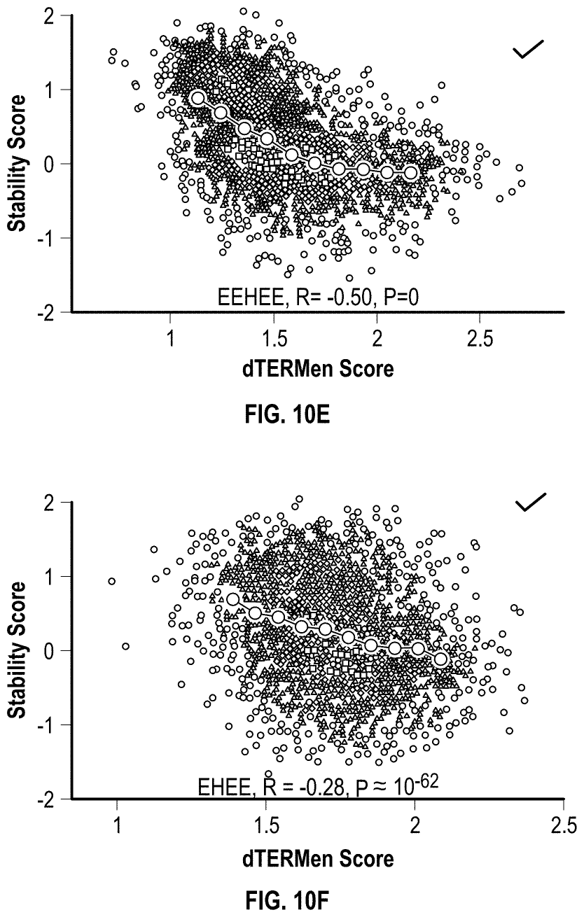

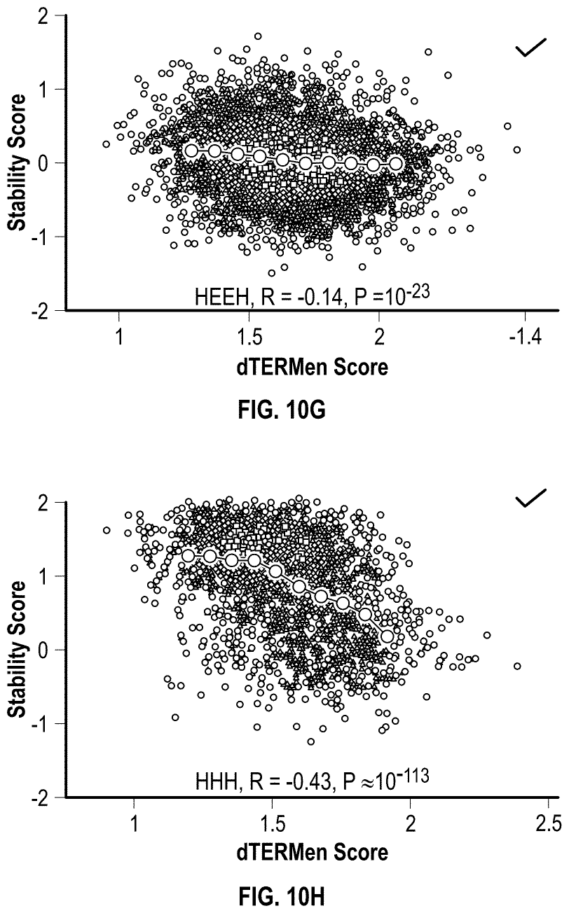

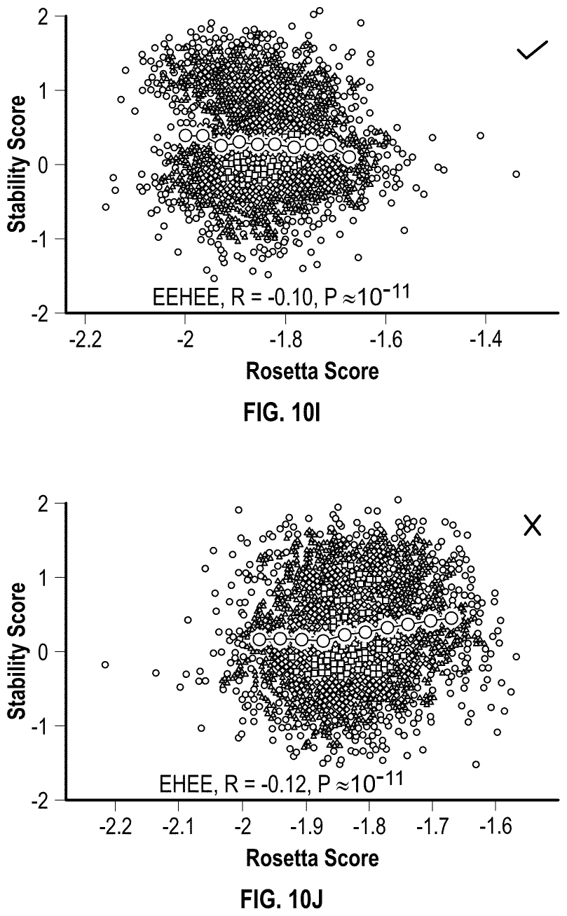

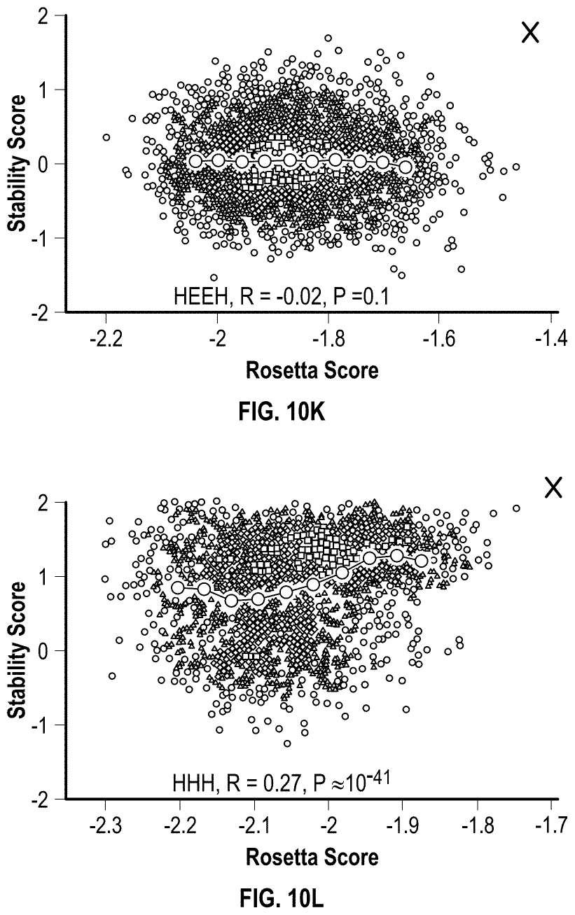

For this example, existing published data on thousands of de-novo designed protein sequences were utilized to determine whether better statistical energy scores tend to indicate higher design success and correlate with better quality of designed proteins. In particular, data published by Baker and co-workers were used, where a total of ˜15,000 de-novo designed sequences for four distinct topologies (see FIG. 10A-10D) were tested, in high throughput, for their ability to form folded, stable, protease-resistant structures (3). Although each of these designs represented a sequence predicted to be well compatible with the desired target backbone by the Rosetta Design software suite (6), most designs failed to fold.

This Example sought to test whether the design methods disclosed herein would better able to distinguish between successful and failed designs. To this end, an exemplary design method was used on each of the ˜15,000 backbone structures deposited by Baker and co-workers (one for each of their designs) (3) to enable the evaluation of any natural amino-acid sequence on any of the target models. An energy score was computed using an exemplary design method disclosed herein for each designed sequence on its respective backbone and divided by sequence length to facilitate comparison across different topologies. FIG. 10E-10H shows, for each of the four topologies, the correlation between the resulting score and the experimental “stability score”—a protease resistance-based metric Baker and co-workers developed to estimate design stability in high throughput, having shown it to correlate closely with thermodynamic stability. Clearly, there was a robust correlation between TERM-based scores and experimental scores (p-values are highly significant in all cases; see legends in FIG. 10E-10H). In contrast, when Rosetta scores computed for each sequence (also published by Baker and colleagues) were considered, the correlation was significantly weaker in all cases (see FIG. 10I-10L). In fact, for three of the four topologies, the correlation coefficient was either statistically insignificant (p-value of 0.1 in FIG. 10K) or of the wrong sign (positive correlation instead of the expected negative in FIGS. 10J and 10L).

Rosetta Design represents the current state of the art in computational protein design (7). Thus, this result indicates that TERM-based scoring synthesizes structure-sequence relationships in a way that cannot be captured by existing design methodologies. Further, the ˜15,000 designed sequences analyzed here were optimized with respect to Rosetta Design and not TERM-based scoring. In fact, TERM-based best-scoring sequences always differed from Rosetta-based designs, on average by 84% (i.e., on average only ˜16% of positions were the same between the Rosetta- and TERM-based-chosen sequences). The ability of the TERM-based methods disclosed herein to quantitatively score even sequences that are different from the optimality region of its own predicted sequence landscape further validates the generality of the method and the universal applicability of the sequence-structure relationships it quantifies.

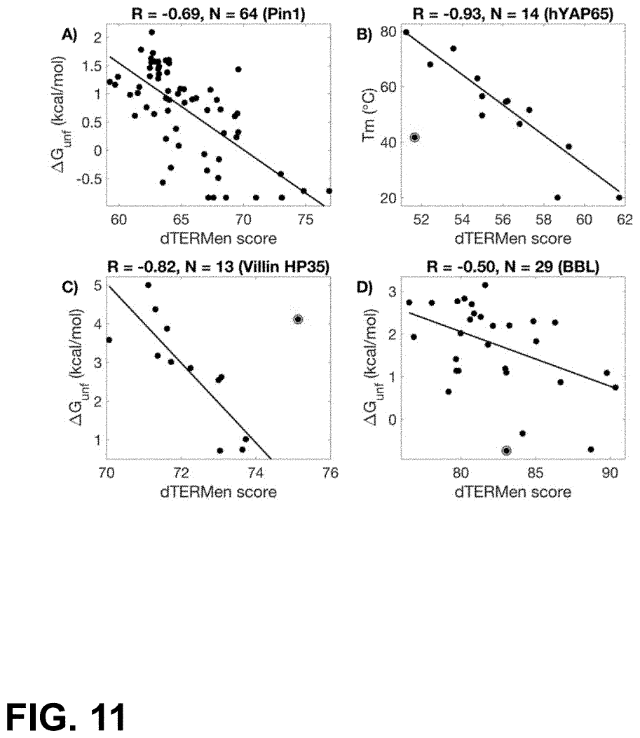

FIG. 11 further shows that the score computed using the exemplary methods disclosed herein correlated closely with thermodynamic stability, using 120 sequence variants of four native domains. These are the same variants that Rocklin et al. used to establish the quantitative nature of their high-throughput experimental stability score (3). The close correlation between TERM-based scores and thermodynamic experiments further validates the TERM-based methodology and suggests that optimization of TERM-based scores is a robust, general-purpose protein design strategy.

### Example 4: Design of a Novel Binding Mode

Protein-protein interactions effectively provide the internal logical wiring of living cells, defining how cells sense and respond to events in and around them. Many cellular protein-protein interactions are encoded by specialized protein-interaction domains. Among these are PDZ domains—modules that specifically bind to C-terminal tails of partner proteins, specifically recognizing the last 6-10 amino acids (8, 9). There are over 250 PDZ domains in the human genome and they are broadly involved in cell signaling and localization (8). Thus, molecules that recognize and inhibit specific PDZ domains represent a great biomedical need. However, because the binding pockets of PDZ domains are structurally conserved, with many domains exhibiting overlapping binding specificities, better inhibition selectivity may be reached if less conserved regions outside the binding pocket are targeted.

This Example utilized two human PDZ domains: the second PDZ domain of protein NHERF-2 (N2P2) and the sixth PDZ domain of protein MAGI-3 (M3P6). Both domains recognize the C-terminus of lysophosphatidic acid receptor 2 (LPA2), and both are implicated in signaling associated with colon cancer (10-13). However, while binding of N2P2 to LPA2 potentiates tumorigenic activities, binding of M3P6 inhibits them (12). Thus, the selective inhibition of N2P2 over M3P6 is relevant as a potential therapeutic route again colon cancer (14).

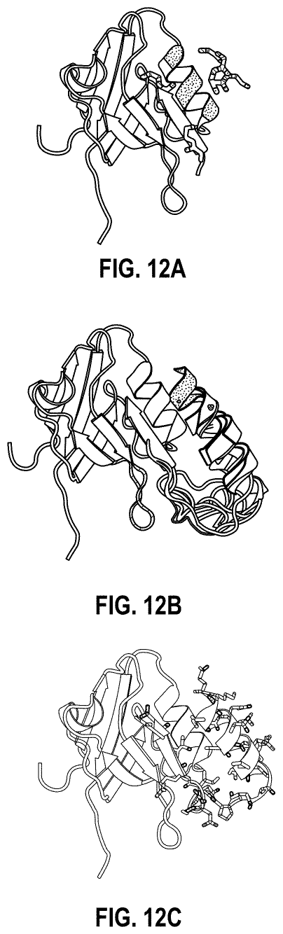

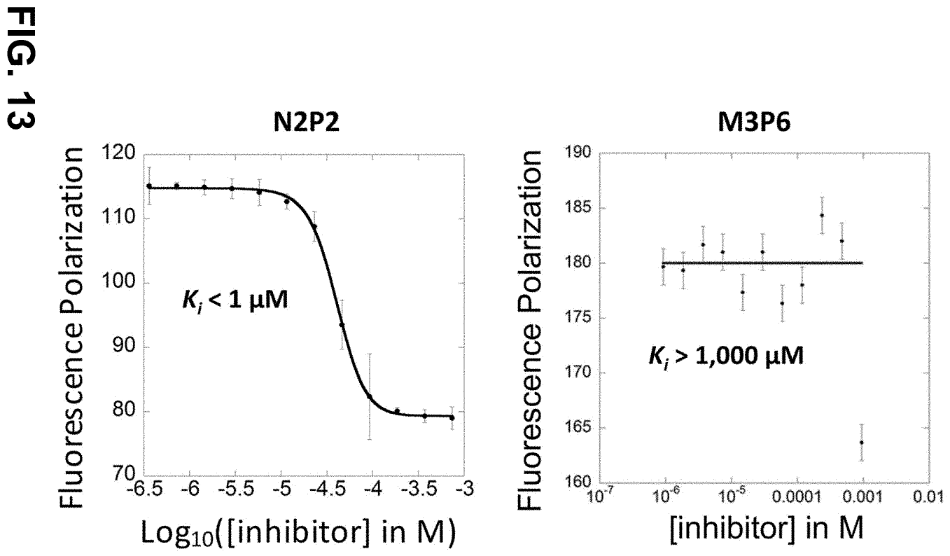

Because both domains natively recognize the same sequence (the C-terminus of LPA2), a TERM-based strategy was employed to extend a known N2P2-binding peptide (taken from the complex structure of N2P2 in PDB entry 2HE4) for making contacts with N2P2 outside of the conserved binding pocket. The strategy identified multi-segment TERMs suitable for completing the existing structure of N2P2—i.e., TERMs with a subset of segments aligning well onto a surface region of N2P2 (interface anchor), the remaining segments forming a putative interface (interface seed), and with TERM sequence statistics compatible with the sequence of the N2P2 anchor region; see FIG. 12. An anchor/seed combination was then manually chosen (based on the N2P2 anchor region mapping to residues not conserved relative to M3P6) and connected with the existing binding peptide by means of intermediate well-overlapping TERMs (see FIG. 12). Finally, the resulting backbone structure, shown in FIG. 12, was subjected to design using an exemplary design method disclosed herein, with the optimal sequence chosen for experimental characterization.

Purified designed peptide was obtained commercially and its affinity to both N2P2 and M3P6 was studied by a Fluorescence Polarization (FP) inhibition assay, as in our previous work (15). FIG. 13 shows that while the affinity towards N2P2 was on the order of 1 μM, there was no detectable interaction with M3P6. By comparison, the C-terminal 6-mer peptide from LPA2 (the native partner for both N2P2 and M3P6) binds ˜30 times weaker to N2P2 while exhibiting approximately equal affinities for N2P2 and M3P6 (15). Thus, the designed novel binding mode shows both improved affinity and drastically improved selectivity.

### Example 5: De-Novo Design of Structures

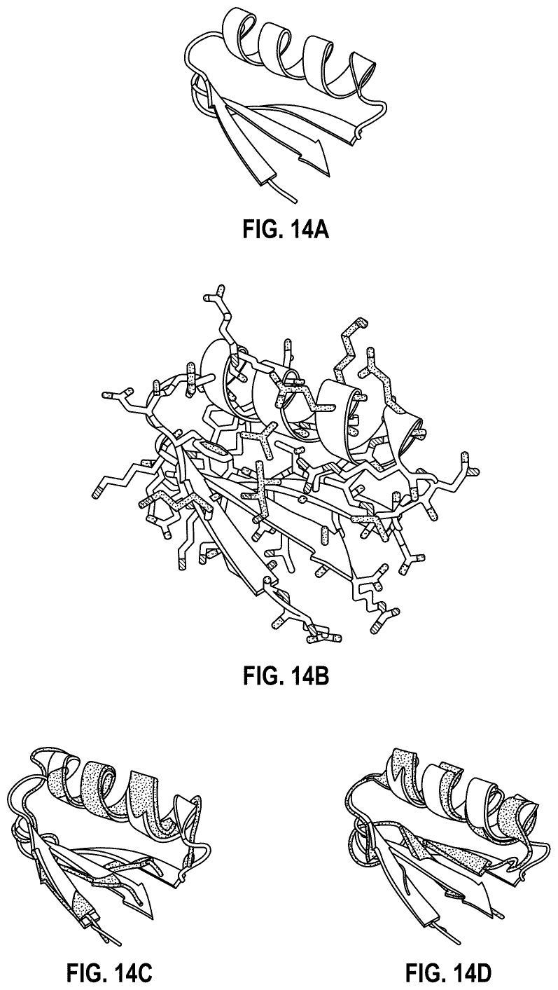

The framework disclosed herein can be applied to arbitrary structures, whether they come from existing protein folds or built de-novo. As an example, FIG. 14A shows a computationally-generated backbone, for which Rocklin and co-workers recently successfully designed a sequence (3). This structure, or any other novel backbone, can be designed via using the methods described above. For this specific backbone, if any natural amino acid was chosen at any of the positions (for a total sequence space of approximately 10⁵²), the solution shown in FIG. 14B was selected optimal. The modeled structure of the designed sequence looked biophysically reasonable (see FIG. 14B). Moreover, submitting the designed sequence to HHpred, a powerful structure-prediction method that relies on the ability to identify remote homologies between the modeled sequence and a protein of known structure (4, 16), revealed PDB entry 5UP5 as the closest match (with a probability of over 97% and alignment coverage of 90%)—the very experimental structure of the corresponding sequence designed by Rocklin et al. (3) (see FIG. 14C). Importantly, 5UP5 was not itself used in the database of proteins queried for TERM-based sequence statistics (and, because it itself a de-novo design, no homologues of it were in the database either). This is strong evidence suggesting that the sequences designed using the exemplary methods disclosed herein have the necessary features such as, for example, likelihood of folding to our target structure. Incidentally, the second match revealed by HHpred, PDB entry 1UTA, is a native structure with a fold highly reminiscent of the target (see FIG. 14D).

### REFERENCES

1. Mackenzie C O, Zhou J, & Grigoryan G (2016) Tertiary alphabet for the observable protein structural universe. Proc Natl Acad Sci USA 113(47):E7438-E7447.
2. Wang H, et al. (2016) LOVTRAP: an optogenetic system for photoinduced protein dissociation. Nat Methods 13(9):755-758.
3. Rocklin G J, et al. (2017) Global analysis of protein folding using massively parallel design, synthesis, and testing. Science 357(6347):168-175.
4. Meier A & Riding J (2015) Automatic Prediction of Protein 3D Structures by Probabilistic Multi-template Homology Modeling. PLoS Comput Biol 11(10):e1004343.
5. Perez-Aguilar J M, et al. (2013) A computationally designed water-soluble variant of a G-protein-coupled receptor: the human mu opioid receptor. PLoS One 8(6):e66009.
6. Leaver-Fay A, et al. (2011) ROSETTA3: an object-oriented software suite for the simulation and design of macromolecules. Methods Enzymol 487:545-574.
7. Alford R F, et al. (2017) The Rosetta All-Atom Energy Function for Macromolecular Modeling and Design. J Chem Theory Comput 13(6):3031-3048.
8. Ivarsson Y (2012) Plasticity of PDZ domains in ligand recognition and signaling. FEBS Lett 586(17):2638-2647.
9. Lee H J & Zheng J J (2010) PDZ domains and their binding partners: structure, specificity, and modification. Cell Commun Signal 8:8.
10. Oh Y S, et al. (2004) NHERF2 specifically interacts with LPA2 receptor and defines the specificity and efficiency of receptor-mediated phospholipase C-beta3 activation. Mol Cell Biol 24(11):5069-5079.
11. Yun C C, et al. (2005) LPA2 receptor mediates mitogenic signals in human colon cancer cells. Am J Physiol Cell Physiol 289(1):C2-11.
12. Lee S J, et al. (2011) MAGI-3 competes with NHERF-2 to negatively regulate LPA2 receptor signaling in colon cancer cells. Gastroenterology 140(3):924-934.
13. Willier S, Butt E, & Grunewald T G (2013) Lysophosphatidic acid (LPA) signalling in cell migration and cancer invasion: a focussed review and analysis of LPA receptor gene expression on the basis of more than 1700 cancer microarrays. Biol Cell 105(8):317-333.
14. Yoshida M, et al. (2016) Deletion of Na+/H+ exchanger regulatory factor 2 represses colon cancer progress by suppression of Stat3 and CD24. Am J Physiol Gastrointest Liver Physiol 310(8):G586-598.
15. Zheng F, et al. (2015) Computational design of selective peptides to discriminate between similar PDZ domains in an oncogenic pathway. J Mol Biol 427 (2):491-510.
16. Zimmermann L, et al. (2017) A Completely Reimplemented MPI Bioinformatics Toolkit with a New HHpred Server at its Core. J Mol Biol.

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, formulations, or methods, or any combination of such changes and modifications of use of the invention, may be made without departing from the spirit and scope thereof.

All references (patent and non-patent) cited above are incorporated by reference into this patent application. The discussion of those references is intended merely to summarize the assertions made by their authors. No admission is made that any reference (or a portion of any reference) is relevant prior art (or prior art at all). Applicant reserves the right to challenge the accuracy and pertinence of the cited references.

What is claimed is:

1. A method for in silico design of an amino acid sequence, comprising the steps of:
decomposing the target structure into a plurality of structural motifs;
identifying, in a structural database, a plurality of structural matches for each of the plurality of structural motifs;
deducing a value for at least one non-local energetic contribution to a sequence-structure relationship using each of the plurality of structural matches; and
generating at least one candidate amino acid sequence, wherein the candidate amino acid sequence possesses a designable property (e.g., is foldable into a binding partner of the target structure).

2. The method of claim 1, wherein the at least one non-local energetic contribution is from a contiguous stretch of backbone around a single design position within one of the plurality of structural motifs.

3. The method of claim 1, wherein the at least one non-local energetic contribution is from a backbone in spatial but not sequence proximity to a single design position within one of the plurality of structural motifs.

4. The method of claim 1, wherein the at least one non-local energetic contribution is from a pair of coupled residues within one of the plurality of structural motifs.

5. The method of any one of claims 1-4, further comprising the step of acquiring a value for at least one local energetic contribution to a sequence-structure relationship using each of the plurality of structural matches.

6. The method of claim 5, wherein the at least one local energetic contribution is from a backbone angle for a single design position within one of the plurality of structural motifs.

7. The method of claim 6, wherein the backbone angle is a phi, psi, or omega angle.

8. The method of any one of claims 1-7, wherein the target structure is a tertiary structure of a protein.

9. The method of any one of claims 1-7, wherein the target structure is a quaternary structure of a protein complex.

10. A method for in silico design of an amino acid sequence, comprising the steps of:
decomposing the target structure into a plurality of structural motifs;
identifying, in a structural database, a plurality of structural matches for each of the plurality of structural motifs;
sequentially deducing a set of values for energetic contributions to a sequence-structure relationship using each of the plurality of structural matches according to a hierarchy of energetic contributions, the hierarchy comprising at least two of:
i. at least one local energetic contribution for a single design position within one of the plurality of structural motifs,
ii. a contiguous stretch of backbone around the single design position,
iii. a backbone in spatial but not sequence proximity to the single design position, and
iv. a pair of coupled residues comprising the single design position; and
generating at least one candidate amino acid sequence that possesses a designable property (e.g., is foldable into a binding partner of the target structure).

11. The method of claim 10, wherein the hierarchy further comprises
v. a triplet of residues comprising the single design position.

12. The method of claim 10 or claim 11, wherein the at least one local energetic contribution is from a backbone angle for a single design position within one of the plurality of structural motifs.

13. The method of claim 10 or claim 11, wherein the at least one local energetic contribution is from a burial state of a single design position within one of the plurality of structural motifs.

14. The method of any one of claims 10-13, wherein the target structure is a tertiary structure of a protein.

15. The method of any one of claims 10-13, wherein the target structure is a quaternary structure of a protein complex.

16. A non-transitory computer-readable storage medium encoded with instructions for in silico design of an amino acid sequence that can fold into a target structure, the instructions executable by a processor and comprising the method of any one of claims 1-15.

17. A method for making a protein that folds into a binding partner of a target structure, comprising:
providing a nucleic acid sequence encoding the candidate amino acid sequence generated in any one of claims 1-15;
introducing the nucleic acid sequence into a host cell; and
expressing the candidate amino acid sequence.

18. The method of claim 17, further comprising determining whether the candidate amino acid sequence folds into the binding partner of the target structure.

19. The method of claim 17, wherein the protein is selected from the group consisting of an enzyme, antibody, receptor, transport protein, hormone, growth factor, and a fragment thereof.

20. A protein produced by the method of any one of claims 17-19.


SEQUENCE LISTING (3 sequences)

SEQ ID NO: 1 - protein, 236 aa, Artificial Sequence
  Red fluorescent protein  derived from Discosoma sp.
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 2 - protein, 236 aa, Artificial Sequence
  TERM-based designed sequence
MVSKGEEDNMAIIKEFMTFEVEMEGTVNGHPFRIRGSGGGDPYEGTQTARLEVVEGGPLPFAWDILSPQFMYGSKAYVKHPADIPDYLKLSFPEGFTWTRTMEFEDGGTVKVTQTSTLKDGKFHYKVKLTGSNFPSDGPVMQKKTMGWEASTERMRPKDGKLEGEIDQELRLKDGGYYRARVRTTYKAKKPVQLPGAYTVRIRLEITSHNEDYTEVEQTETAKGEHSTGGMDELYK

SEQ ID NO: 3 - protein, 40 aa, Artificial Sequence
  TERM-based designed sequence
EATKEFDGPEEAEKVKKELEERNLEVEVEKKDGKYKVTAR